(19) United States
(12) Patent Application Publication
(10) Pub. No.: US 2010/0292231 A1
(43) Pub. Date: Nov. 18, 2010

(54) Indazole Compounds for Treating Inflammatory Disorders, Demyelinating Disorders and Cancers

(76) Inventors: Alfred M. Ajami, Brookline, MA (US); Kenneth Duncan, Waltham, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/698,597

(22) Filed: Feb. 2, 2010

Related U.S. Application Data
(63) Continuation-in-part of application No. PCT/US2008/009263, filed on Jul. 30, 2008.
(60) Provisional application No. 60/963,144, filed on Aug. 2, 2007.

(51) Int. Cl. A61K 31/5377; C07D 471/16; A61K 31/4745; C07D 413/14; C07D 401/14; A61K 31/497; A61P 35/00; A61P 29/00; A61P 11/00; A61P 17/06; A61P 31/18

(52) U.S. Cl. 514/233.2; 546/48; 514/283; 544/125; 544/361; 514/253.02

(57) ABSTRACT

Compounds of formula (I) or formula (Ia) and a method of treating a patient suffering from certain inflammatory disorders, demyelinating disorders, FLT3-mediated disorders, CSF-1R-mediated disorders, cancers and leukemias, comprising administering to said patient a therapeutically effective amount of a compound of formula (I) or formula (Ia) or a pharmaceutically acceptable salt thereof.

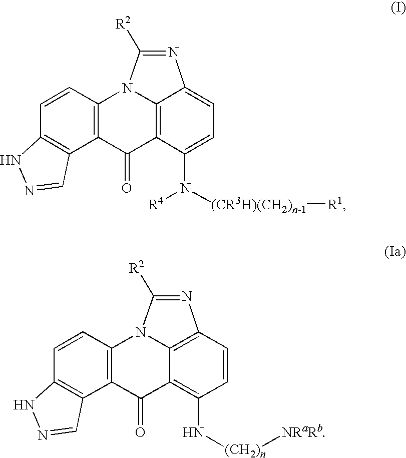

Definitions for the variables are provided herein.

### RELATED APPLICATIONS

This application is a continuation-in-part of International Application No. PCT/US2008/009263, which designated the United States and was filed on 30 Jul. 2008, published in English, which claims the benefit of U.S. Provisional Application No. 60/963,144, filed Aug. 2, 2007. The entire teachings of the above applications are incorporated herein by reference.

### BACKGROUND OF THE INVENTION

There is a need for new pharmaceutically acceptable therapies for an inflammatory disorder, a demyelinating disorder, a FLT3-mediated disorder, a cancer, a leukemia or a CSF-1R-mediated disorder in a patient.

### SUMMARY OF THE INVENTION

The present invention is directed to a class of novel compounds that can be used for treatment of an inflammatory disorder, a demyelinating disorder, a FLT3-mediated disorder, a cancer, a leukemia, MYLK2-mediated barrier dysfunction disorder, or a CSF-1R-mediated disorder in a patient.

In one embodiment, the present invention is a compound of formula (I) or a pharmaceutically acceptable salt thereof, or a compound of formula (la) or a pharmaceutically acceptable salt thereof:

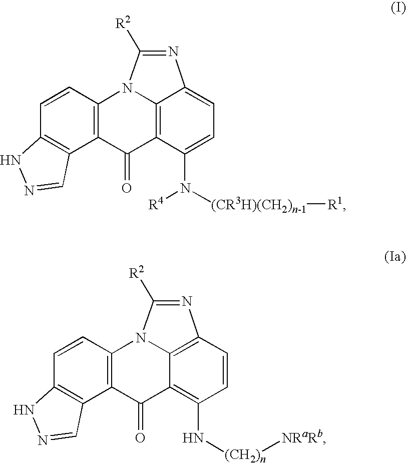

wherein R1 is H or NRaRb; Ra and Rb, each independently are hydrogen or an optionally substituted alkyl; or Ra and Rb, taken together with the nitrogen to which

they are attached, form a non-aromatic heterocycle, optionally substituted at one or more substitutable carbon atoms with methyl, hydroxyl, or methoxy, and optionally N′-substituted with C1-C4 alkyl or C1-C4 alkyl substituted with —NRcRd; wherein Rc and Rd are individually H, methyl or ethyl; R2 is H or C1-C4 alkyl; R3 is H or C1-C4 alkyl; n is an integer from 2 to 5 and R4 is H or C1-C4 alkyl.

In another embodiment, the present invention is a method of treating an inflammatory disorder, a demyelinating disorder, a FLT3-mediated disorder, a cancer, a leukemia or a CSF-1R-mediated disorder in a patient, comprising administering to said patient a therapeutically effective amount of a compound.

In another embodiment, the present invention is a pharmaceutical composition comprising a therapeutically effective amount of a compound of formula (I) or formula (Ia) or a pharmaceutically acceptable salt thereof in a pharmaceutically acceptable carrier.

In another embodiment, the present invention is a use of a compound of formula (I) or formula (Ia) for the manufacture of a medicament for treatment of an inflammatory disorder, a demyelinating disorder, a FLT3-mediated disorder, a cancer, a leukemia or a CSF-1R-mediated disorder in a patient.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a bar plot of percent cell migration into the scratch in each sample (measured with respect to the control).

FIG. 2 is a plot of percent confluence of the cells in the wound (scratch) as a function of time (measured with respect to the control).

FIG. 3A is a plot of percent invasion as a function of concentration of the control compound (Symadex®).

FIG. 3B is a plot of percent invasion as a function of concentration of the indicated test compound.

FIG. 3C is a plot of percent invasion as a function of concentration of the indicated test compound.

FIG. 3D is a plot of percent invasion as a function of concentration of the indicated test compound.

FIG. 4 is a bar plot of absorbance of the cell cultures subjected to the BrdU assay, each sample including the specified test compound.

FIG. 5 is a bar plot of the number of cell in each sample containing more than three nuclei (osteoclast differentiation assay). Each sample including the specified test compound at the specified concentration.

### DETAILED DESCRIPTION OF THE INVENTION

It has now been discovered that administration of certain indazole compounds can be used to treat an inflammatory disorder, a demyelinating disorder, a FLT3-mediated disorder, a cancer, a leukemia or a CSF-1R-mediated disorder in a patient.

In one embodiment, the present invention is a compound of formula (I) below or a pharmaceutically acceptable salt thereof or compound of formula (Ia) or a pharmaceutically acceptable salt thereof.

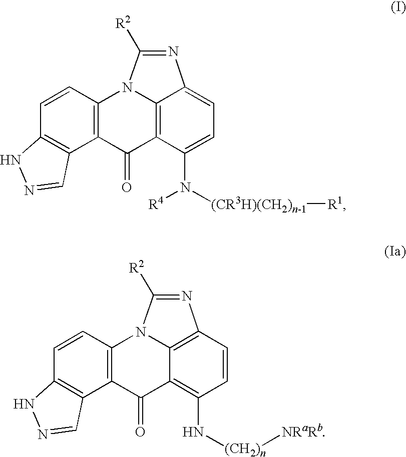

Values and preferred values for the variables in formula (I) or formula (Ia) are defined below.

R¹ is H or NR^{a}R^{b}. In one embodiment R¹ is H, alternatively R¹ is NR^{a}R^{b}.

R² is H or an optionally substituted C1-C4 alkyl. Preferably, R² is H.

R³ is H or C1-C4 alkyl; preferably, R³ is methyl.

R⁴ is H or C1-C4 alkyl; preferably, R⁴ is methyl or ethyl.

Integer n is from 2 to 5, preferably, 2 or 3. In another embodiment, n is 2; alternatively, n is 3. In another embodiment, n is 4; alternatively, n is 5.

R^{a} and R^{b}, each independently, are hydrogen or an optionally substituted alkyl, Alternatively, R^{a} and R^{b}, taken together with the nitrogen to which they are attached, form a non-aromatic heterocycle, each optionally substituted at one or more substitutable carbon atoms with methyl, hydroxyl, or methoxy, and optionally N′-substituted with C1-C4 alkyl or C1-C4 alkyl substituted with —NR^{c}R^{d}.

Preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Alternatively, R^{a} and R^{b} or individually are hydrogen or a C1-C3 alkyl optionally substituted with —OH, —SH, halogen, cyano, nitro, amino, —COOH, a C1-C3 alkyl.

More preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}.

More preferably, R^{a} and R^{b}, taken together with the nitrogen to which they are attached, form a 5-7 member non-aromatic heterocycle selected form a group consisting of:

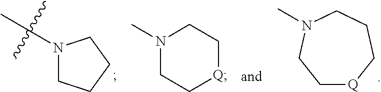

Q is S, O, CH₂, NH, or NR¹⁰², and R¹⁰² is methyl or ethyl. More preferably, R^{a} and R^{b}, taken together with the nitrogen to which they are attached, form N-morpholinyl or N-piperidinyl.

More preferably, R^{a} and R^{b} individually are hydrogen or a C1-C3 alkyl optionally substituted with —OH, —SH, halogen, cyano, nitro, amino, —COOH, a C1-C3 alkyl. More preferably. R^{a} and R^{b} individually are H, methyl or ethyl.

R^{c} and R^{d} are individually H, methyl or ethyl.

In a first preferred embodiment, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C 1-C4 alkyl substituted with —NR^{c}R^{d}; or R^{a} and R^{b} or both are hydrogen or a C1-C3 alkyl optionally substituted with —OH, —SH, halogen, cyano, nitro, amino, —COOH, a C1-C3 alkyl. Preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Values and preferred values for the remainder of variables are as defined above in formula (I) and (Ia).

In another embodiment, the compound of the invention is represented by formula (II) or a formula (IIa):

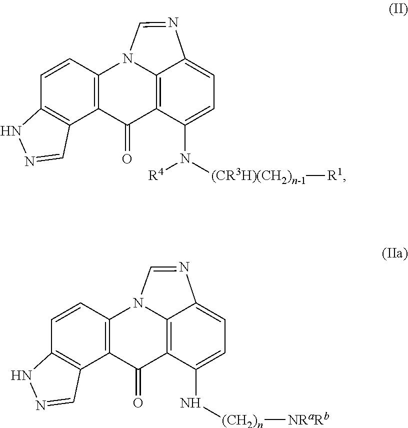

Values and preferred values for formula (II) and (IIa) are as described above for formula (I) and (Ia).

In another embodiment, the compound of the invention is represented by formula (II) or formula (IIa), wherein: R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Values and preferred values for the remainder of variables are as defined above in formula (I) and (Ia).

In another embodiment, the compound of the invention is represented by formula (II), or formula (IIa), wherein: n is 2 or 3; and R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, and values and preferred values for the remainder of variables are as defined above in formula (I) and (Ia). Preferably, R^{a} and R^{b}, taken together with the nitrogen to which they are attached, form a 5-7 member non-aromatic heterocycle selected form a group consisting of:

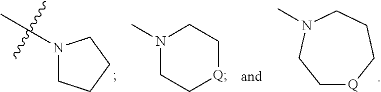

wherein Q is S, O, CH₂, NH, or NR¹⁰², and R¹⁰² is methyl or ethyl. More preferably, R^{a} and R^{b}, taken together with the nitrogen to which they are attached, form N-morpholinyl or N-piperidinyl.

In another embodiment, the compound of the invention is represented by formula (II) or formula (IIa), wherein: R^{a} and R^{b} individually are hydrogen or a C1-C3 alkyl optionally substituted with —OH, —SH, halogen, cyano, nitro, amino, —COOH, or C1-C3 alkyl; and the values and preferred values for the remainder of the variables are as described for formula (I) and (Ia).

In another embodiment, the compound of the invention is represented by formula (II) or formula (IIa), wherein: n is 2 or 3 and R^{a} and R^{b} individually are hydrogen or a C1-C3 alkyl optionally substituted with —OH, —SH, halogen, cyano, nitro, amino, —COOH, or C1-C3 alkyl; and preferred values for the variables are as described for formula (I). More preferably, R^{a} and R^{b} both are H, methyl or ethyl.

Alternatively, for embodiments described in the previous seven paragraphs, R¹ is H and the reminder of the variables are as described above. In another alternative, in the embodiments described in the previous seven paragraphs, R¹ is NR^{a}R^{b} and the reminder of the variables are as described.

Examples of compounds of the invention include:

| Compound No. | Structure |
|---|---|
| (III) | 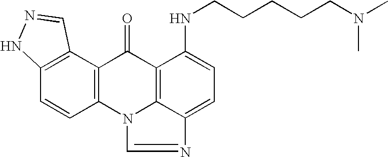 |
| (IV) | 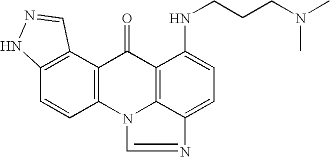 |
| (V) | 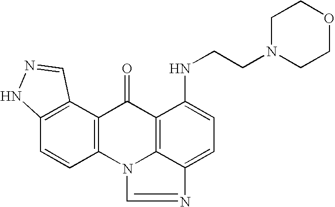 |
| (VI) | 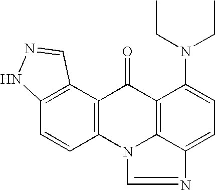 |
| (VII) | 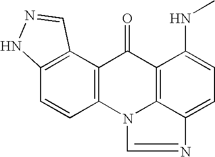 |
| (VIII) | 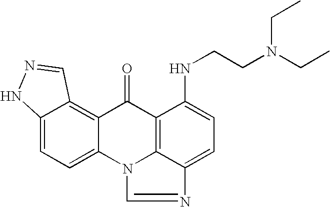 |
| (IX) | 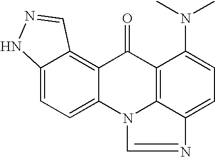 |
| (X) | 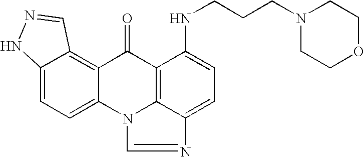 |
| (XI) | 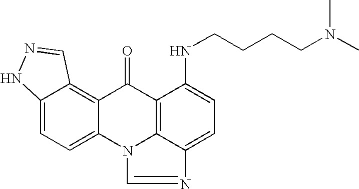 |
| (XII) | 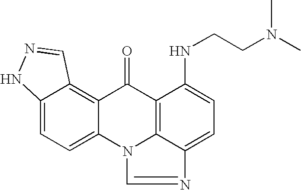 |
| (XIV) | 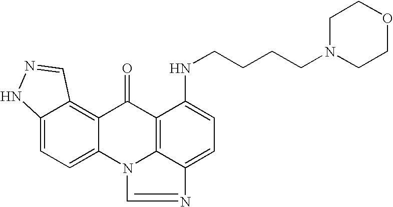 |
| (XV) | 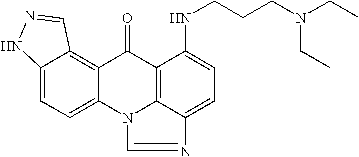 |
| (XVI) | 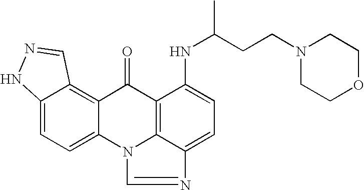 |
| (XVII) | 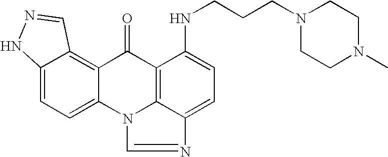 |

The term “alkyl”, as used herein, unless otherwise indicated, includes straight or branched saturated monovalent hydrocarbon radicals, typically C1-C10, preferably C1-C6. Examples of alkyl groups include, but are not limited to, methyl, ethyl, propyl, isopropyl, and t-butyl. Suitable substituents for a substituted alkyl include —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, where p and q are independently an integer from 1 to 6.

The term “haloalkyl”, as used herein, includes an alkyl substituted with one or more F, Cl, Br, or I, wherein alkyl is defined above.

The terms “alkoxy”, as used herein, means an “alkyl-O—” group, wherein alkyl, is defined above.

The term “haloalkoxy”, as used herein, means “haloalkyl-O—”, wherein haloalkyl is defined above.

As used herein, an amino group may be a primary (—NH₂), secondary (—NHRₓ), or tertiary (—NRₓR_{y}), wherein Rₓ and R_{y} may be any of the optionally substituted alkyls described above.

The term “non-aromatic heterocycle” refers to non-aromatic carbocyclic ring systems typically having four to eight members, preferably five to six, in which one or more ring carbons, preferably one to four, are each replaced by a heteroatom such as N, O, or S. Non-aromatic heterocycles can be optionally unsaturated. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrorolidinyl, 1-piperazinyl, 2-piperazinyl, 1-piperidinyl, 2-piperidinyl, 3-piperidinyl, 4-piperidinyl, 4-thiazolidinyl, diazolonyl, and N-substituted diazolonyl.

The non-aromatic heterocyclic group may be C-attached or N-attached (where such is possible). For instance, a group derived from pyrrole may be pyrrol-1-yl (N-attached) or pyrrol-3-yl (C-attached).

Suitable substituents for a non-aromatic heterocyclic group are those that do not substantially interfere with the pharmaceutical activity of the disclosed compound. One or more substituents can be present, which can be identical or different. Examples of suitable substituents for a substitutable carbon atom in aryl, heteroaryl or a non-aromatic heterocyclic group include —OH, halogen (—F, —Cl, —Br, and —I), —R′, haloalkyl, —OR′, —CH₂R′, —CH₂OR′, —CH₂CH₂OR′, —CH₂OC(O)R′, —O—COR′, —COR′, —SR′, —SCH₂R′, —CH₂SR′, —SOR′, —SO₂R′, —CN, —NO₂, —COOH, —SO₃H, —NH₂, —NHR′, —N(R′)₂, —COOR′, —CH₂COOR′, —CH₂CH₂COOR′, —CHO, —CONH₂, —CONHR′, —CON(R′)₂, —NHCOR′, —NR′COR′, —NHCONH₂, —NHCONR′H, —NHCON(R′)₂, —NR′CONH₂, —NR′CONR′H, —NR′CON(R′)₂, —C(═NH)—NH₂, —C(═NH)—NHR′, —C(═NH)—N(R′)₂, —C(═NR′)—NH₂, —C(═NR′)—NHR′, —C(═NR′)—N(R′)₂, —NH—C(═NH)—NH₂, —NH—C(═NH)—NHR′, —NH—C(═NH)—N(R′)₂, —NH—C(═NR′)—NH₂, —NH—C(═NR′)—NHR′, —NH—C(═NR′)—N(R′)₂, —NR′H—C(═NH)—NH₂, —NR′—C(═NH)—NHR′, —NR′—C(′NH)—N(R′)₂, —NR′—C(═NR′)—NH₂, —NR′—C(═NR′)—NHR′, —NR′—C(═NR′)—N(R′)₂, —SO₂NH₂, —SO₂NHR′, —SO₂NR′₂, —SH, —SOₖR′ (k is 0, 1 or 2) and —NH—C(═NH)—NH₂. Each R′ is independently an alkyl group. Oxo (C══O) and thio (C══S) are also suitable substituents for a non-aromatic heterocycle.

Suitable substituents on the nitrogen of a non-aromatic heterocyclic group include —R″, —N(R″)₂, —C(O)R″, —CO₂R″, —C(O)C(O)R″, —C(O)CH₂C(O)R″, —SO₂R″, —SO₂N(R″)₂, —C(═S)N(R″)₂, —C(═NH)—N(R″)₂, and —NR″SO₂R″. R″ is hydrogen, an alkyl or alkoxy group.

At various places in the present specification, substituents of compounds are disclosed in groups or in ranges. It is specifically intended that the description include each and every individual subcombination of the members of such groups and ranges. For example, the term “C1-6 alkyl” is specifically intended to individually disclose C1, C₂, C₃, C₄, C₅, C₆, C1-C₆, C1-C₅, C1-C₄, C1-C₃, C1-C₂, C₁₋-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-7 member non-aromatic heterocycle” is specifically intended to individually disclose a heterocycle having 5, 6, 7, 5-7, 5-6, and 6-7 ring atoms.

Throughout the specification, structures may or may not be presented with chemical names. Where any question arises as to nomenclature, the structure prevails.

### Disorders Treatable by the Compounds of the Invention

It has now been discovered, that compounds of formula (I) or formula (Ia) or formula (II) or formula (IIa) can be used to treat an inflammatory disorder, a demyelinating disorder, a FLT3-mediated disorder, a cancer, a leukemia or a CSF-1R-mediated disorder in a patient. The term “patient” means a warm blooded animal, i.e., a mammal, such as for example rat, mice, dogs, cats, guinea pigs, and primates including humans. The terms “treat” or “treating” include any treatment, including, but not limited to, alleviating symptoms, eliminating the causation of the symptoms either on a temporary or permanent basis, or preventing or slowing the appearance of symptoms and progression of the named disorder or condition.

### 1. Cancers

In one embodiment, the present invention is a method of treating a patient suffering from a cancer. The method comprises administering to a patient a therapeutically effective amount of a compound of formula (I) or formula (Ia) or formula (II) or formula (IIa) or a pharmaceutically acceptable salt thereof.

In another embodiment, the present invention is a method of treating a subject suffering from a cancer. As used herein, the term “cancer” refers to the uncontrolled growth of abnormal cells that have mutated from normal tissues. A cancerous tumor (malignancy) is of potentially unlimited growth and expands locally by invasion and systemically by metastasis. Examples of cancers that can be treated by the compounds of the present invention include: breast cancer, colorectal cancer, non-small cell lung cancer, ovarian, renal, sarcoma, melanoma, head & neck, hepatocellular, thyroid, multidrug-resistant leukemia, lymphoma, multiple myeloma, esophageal, large bowel, pancreatic, mesothelioma, carcinoma (e.g. adenocarcinoma, including esophageal adenocarcinoma), sarcoma (e.g. spindle cell sarcoma, liposarcoma, leiomyosarcoma, abdominal leiomyosarcoma, sclerosing epithelioid sarcoma) and melanoma (e.g. metastatic malignant melanoma). In one embodiment, the patient can be treated for bone metastases. Treatment of subtypes of the aforementioned cancers is also included. Subtypes are described in the following paragraphs.

“Treating a subject suffering from cancer” includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

“Treating a bone metastases”, as used herein, refers to reducing (partially or completely) the size of the bone metastases, slowing the growth of the metastases relative to the absence of treatment and reducing the extent of further spread of the cancer. “Treating a bone metastases” also includes pain reduction, decreased incidents of fractures, relief of spinal cord compression, control of hypercalcaemia, and/or restoration of normal blood cell counts.

“Breast cancer” includes, but is not limited to, ductal carcinoma, lobular carcinoma, inflammatory carcinoma, medullary carcinoma, colloid or mucinous carcinoma, papillary carcinoma, tubular carinoma, triple negative breast cancer, inflammatory breast cancer, metaplastic carcinoma, Paget's disease, and Phyllodes tumor.

As used herein “ovarian cancer”, is cancer of the ovaries or fallopian tubes, including cancers of germ cells, stromal cells, and epithelial cells. Examples of ovarian cancers include but are not limited to:

Epithelial Ovarian Tumors, which include but are not limited to, serous adenomas, mucinous adenomas, and Brenner tumors, tumors of low malignant potential (LMP tumors), borderline epithelial ovarian cancer, epithelial ovarian cancers, carcinomas and undifferentiated epithelial ovarian carcinomas;

Germ Cell tumors which include but are not limited to, teratoma, dysgerminoma, endodermal sinus tumor, and choriocarcinoma; and

Stromal tumors, which include but are not limited to, granulosa cell tumors, granulosa-theca tumors, and Sertoli-Leydig cell tumors.

“Renal cancer” or “kidney cancer”, as used herein, includes but is not limited to, transitional cell cancer (TCC) of the renal pelvis. Wilms Tumour and renal cell cancer.

Renal cell cancer is also called renal adenocarcinoma or hypernephroma. In renal cell cancer, the cancerous cells are found in the lining of the tubules (the smallest tubes inside the nephrons that help filter the blood and make urine).

There are several types of renal cell cancer including but not limited to clear cell, chromophilic, chromophobic, oncocytic, collecting duct and sarcomatoid.

Renal cancer also includes cancers containing more than one of the cell types described above.

As used herein. “melanoma” is a type of skin cancer that occurs in the cells that color the skin, called melanocytes. Types of melanoma include but are not limited to:

Cutaneous melanoma, superficially spreading melanoma, nodular malignant melanoma, lentiginous malignant melanoma, acral lentiginous melanoma, demoplastic malignant melanomas, giant melanocytic nevus, amelanotic malignant melanoma, acral lentiginous melanoma unusual melanoma variants, including mucosal malignant melanoma and ocular malignant melanoma.

“Sarcomas”, as used herein, include but are not limited to, fibrosarcomas from fibrous body tissues, leiomyosarcomas and rhabdomyosarcomas from muscle tissues, liposarcomas from fat, synovial sarcomas, angiosarcomas from blood vessels, MPNST—malignant peripheral nerve sheath tumours (PNSTs), GIST—gastrointestinal stromal sarcoma, osteosarcoma, myosarcoma, chondrosarcoma, bile duct sarcoma, brain sarcoma, breast sarcoma, soft tissue sarcoma, uterine sarcoma, endocardial sarcoma, stromal sarcomas from supporting tissues (endometrial stromal sarcoma), granuloytic, histiolytic, hemangioendothelial, Kupffer-cell, neurogenic, round-cell, reticulum cell, spindle cell, Kaposi's sarcoma of the skin, Ewing's sarcomas and PNETs. In certain embodiments, the sarcoma is leiomyosarcoma or liposarcoma.

“Thyroid cancer”, as used herein, includes but is not limited to, papillary and/or mixed papillary/follicular, follicular and/or Hurthle cell, lymphoma, medullary, anaplastic and combinations thereof.

The term “head and neck cancer” as used herein, encompasses tumors that occur in several areas of the head and neck region, including the nasal passages, sinuses, mouth, throat, larynx (voice box), swallowing passages, salivary glands, and skin cancers that develop on the scalp, face, or neck may also be considered head and neck cancers. These cancers include but are not limited to squamous cell carcinoma, mucoepidermoid carcinoma, adenoid cystic carcinoma, lymphoma, adenocarcinoma, esthesioneuroblastoma, tumors of the nasal cavity and paranasal sinuses, nasopharyngeal cancer, cancers of the oral cavity (including all the various parts of the mouth: the lips; the lining inside the lips and cheeks (the buccal mucosa); the bottom of the mouth; the front of the tongue; the front part of the top of the mouth (the hard palate); the gums; and the area behind the wisdom teeth (the retromolar trigone)), tumors of the oropharynx, hypopharyngeal tumors, laryngeal cancer and salivary gland cancer (including malignant salivary gland tumor).

As used herein “hepatocellular cancer” or “liver cancer” includes but is not limited to: hepatocellular carcinoma (also sometimes called hepatoma or HCC) “carcinoma”, fibrolamellar HCC, cholangiocarcinoma, angiosarcoma (also be called haemangiosarcoma) and hepatoblastoma.

As used herein, “non-small cell lung cancer” includes, squamous cell carcinoma, adenocarcinoma and undifferentiated non-small cell lung cancer (undeveloped cancer cells are known as undifferentiated cells) and large cell carcinoma.

“Colorectal cancer” as used herein, includes any type of colon or rectal cancer, including but not limited to, adenoscarcinoma, sarcoma, melanoma, stromal, carcinoid, and lymphoma.

### 2. Inflammatory Conditions

In one embodiment, the present invention is a method of treating a patient suffering from an inflammatory condition. The method comprises administering to a patient a therapeutically effective amount of a compounds of formula (I) or formula (Ia) or formula (II) or formula (IIa) or a pharmaceutically acceptable salt thereof. The condition can be systemic lupus, psoriasis, Crohn's disease, inflammatory bowel disease (IBD), ulcerative colitis, rheumatoid arthritis, sarcoid, Alzheimer's disease, insulin dependent diabetes mellitus, atherosclerosis, asthma, spinal cord injury, stroke, a chronic inflammatory demyelinating neuropathy, multiple sclerosis, a congenital metabolic disorder, a neuropathy with abnormal myelination, drug-induced demyelination, radiation induced demyelination, a hereditary demyelinating condition, a prion-induced demyelination, encephalitis-induced demyelination.

Examples of chronic inflammatory demyelinating neuropathies include: Chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

### 3. Demyelinating Conditions

In another embodiment, the present invention is a method of treatment of a patient suffering from a demyelinating condition. The method comprises administering to a patient a therapeutically effective amount of a compound of formula (I) or formula (Ia) or formula (II) or formula (IIa) or a pharmaceutically acceptable salt thereof. As used herein, a “demyelinating condition” is a condition that destroys, breaks the integrity of or damages a myelin sheath. As used herein, the term “myelin sheath” refers to an insulating layer surrounding vertebrate peripheral neurons, that increases the speed of conduction and formed by Schwann cells in the peripheral or by oligodendrocytes in the central nervous system. Such condition can be multiple sclerosis, a congenital metabolic disorder, a neuropathy with abnormal myelination, drug-induced demyelination, radiation induced demyelination, a hereditary demyelination condition, a prion-induced demyelination, encephalitis-induced demyelination, a spinal cord injury, Alzheimer's disease as well as chronic inflammatory demyelinating neuropathies, examples of which are given above. In one embodiment, the condition is multiple sclerosis. The method comprises administering to a patient a therapeutically effective amount of a compound of formula (I) or formula (Ia) or formula (II) or formula (IIa) or a pharmaceutically acceptable salt thereof.

In another embodiment, the present invention is a method of promoting remyelination of nerve cells in a patient, comprising administering to the patient in need thereof a therapeutically effective amount of a compound of formula (I) or formula (Ia). The patient can be suffering from any of the demyelinating conditions listed above.

In another embodiment, the present invention is a method of preventing demyelination and promoting remyelination in a patient in need thereof, comprising administering a combination of a therapeutically effective amount of a compound of formula (I) or formula (Ia) or formula (II) or formula (IIa) or pharmaceutically acceptable salt thereof, and an anti-inflammatory agent as described below.

In another embodiment, the present invention is a method of reversing paralysis in a subject in need thereof with a demyelinating disease, comprising administering to the subject a compound in an amount sufficient to inhibit lymphocyte infiltration of immune cells in the spinal cord to promote remyelination of nerve cells in the spinal cord and thereby treating paralysis in said subject, wherein the compound is of formula (I) or formula (Ia) or formula (II) or formula (IIa) or a pharmaceutically acceptable salt thereof.

Diseases such as, for example, multiple sclerosis (MS), systemic lupus erythematosus (SLE), rheumatoid arthritis (RA), inflammatory bowel disease (IBD) and psoriasis are considered autoimmune diseases which represent assaults by the body's immune system which may be systemic in nature, or else directed at individual organs in the body. They appear to be diseases in which the immune system makes mistakes and, instead of mediating protective functions, becomes the aggressor. In one aspect, the compounds of the present invention are used for the treatment of autoimmune diseases.

The compounds of the present invention can be used in models for the treatment of autoimmune diseases such as multiple sclerosis and rheumatoid arthritis. One method of showing the utility of a pharmaceutical compound for the treatment of various conditions associated with multiple sclerosis (MS) is its ability to inhibit effects of experimental allergic encephalomyelitis in laboratory animals.

Experimental allergic encephalomyelitis (EAE) is an animal model for MS, which entails inducing a T-cell-mediated autoimmune disease against myelin basic protein in certain susceptible mammalian species. The EAE model is an appropriate method for studying the inflammation of the brain and spinal cord associated with MS (see Bolton, C. Mult, Scler, 1995; 1(3);143-9).

In rodents, injection of whole spinal cord or spinal cord components such as myelin basic protein induces an autoimmune response based on the activation of T-lymphocytes. Clinical disease typically becomes manifest around day 8-10 after inoculation, observed as a broad spectrum of behavioral anomalies ranging from mild gait disturbances and tail atony to complete paralysis and death. Weight loss typically occurs. In animals that survive, spontaneous recovery occurs, accompanied by variable recovery of most motor function. Depending on the species, allergen, and methodology used, animals tested by the EAE model may experience a single (acute EAE) or several (chronic relapsing EAE) attacks.

Treatments of EAE come in many structural forms: treatment can be prophylactic or preventative, whereby the therapeutic composition is administered before immunization; treatment can be initiated during the first week of induction; and treatment can be interventious, initiated after clinical symptoms are extent (acute or chronic). Prevention protocols are very common in the literature, treatment after disease is rarer, and treatment after weeks of disease are the most infrequent.

Rheumatoid Arthritis (RA) is an autoimmune disorder characterized by the chronic erosive inflammation in joints leading to the destruction of cartilage and bones. Several disease modifying antirheumatic drugs (DMARDS) are used in the treatment of RA. Currently, the two most important DMARDS are inhibitors of tumor necrosis factor α (TNF-α) and methotrexate (MTX). One method for demonstrating the utility of a pharmaceutical compound for the treatment of various conditions associated with RA is its ability to inhibit the induction of arthritis by collagen monoclonal antibodies (mABs) in mice.

Collagen-induced Arthritis (CIA) is an experimental autoimmune disease that can be elicited in susceptible strains of rodents (rat and mouse) and nonhuman primates by immunization with type II collagen, the major constituent protein of articular cartilage. CIA manifests as swelling and erythema in the limbs of the mouse. This model of autoimmunity shares several clinical and pathological features with rheumatoid arthritis (RA) and has become the most widely studied model of RA. CIA in the mouse model was first described by Courtenay et al. in 1980 (Courtnay, J. S., Dallman, M. J., Dayman, A. D., Martin A., and Mosedale, B. (1980) Immunisation against heterologous type II collagen induces arthritis in mice. Nature 283, 666-668). Like RA, susceptibility to CIA is regulated by the class II molecules of the major histocompatibility complex (MHC), indicating the crucial role played by T cells.

### 4. FLT3-Mediated Disorders

In one embodiment, the present invention is a method of treating a patient suffering from a FLT3-mediated disorder. The method comprises administering to a patient a therapeutically effective amount of a compound of formula (I) or formula (Ia) or formula (II) or formula (IIa) or a pharmaceutically acceptable salt thereof.

As used herein, the term “FLT3-mediated disorder” is a disorder in which one or more symptoms can be inhibited, alleviated, reduced or whose onset can be delayed by inhibiting completely or partially the FLT3 protein kinase. Inhibition of FLT3 has been shown to suppress immune response, possibly via inhibition of DC-induced stimulation of T cells, and may be considered for the treatment of autoimmune diseases (see, WO 2006/020145 A2; Whartenby, et al, PNAS, 2005, 102, 16741-16746).

The terms “treat” or “treating”, when used with reference to a FLT3-mediated condition, include any treatment, including, but not limited to, alleviating symptoms, eliminating the causation of the symptoms associated with a FLT3-mediated condition either on a temporary or permanent basis, or preventing or slowing the appearance of symptoms and progression of the named disorder or condition.

As used herein the term “therapeutically effective amount”, when used with reference to a FLT3-mediated condition, is the amount of a compound disclosed herein that will achieve a partial or total inhibition or delay of the progression of a FLT3-mediated disorder in a patient.

FLT3-mediated disorders and conditions include axonal degeneration, acute transverse myelitis, amyotrophic lateral sclerosis, infantile spinal muscular atrophy, juvenile spinal muscular atrophy, Creutzfeldt-Jakob disease, subacute sclerosing panencephalitis, organ rejection, bone marrow transplant rejection, non-myeloablative bone marrow transplant rejection, ankylosing spondylitis, aplastic anemia, Behcet's disease, graft-versus-host disease, Graves' disease, autoimmune hemolytic anemia, Wegener's granulomatosis, hyper IgE syndrome, idiopathic thrombocytopenia purpura, and Myasthenia gravis.

### 5. Leukemias

In one embodiment, the compounds of the present invention, for example the compounds of formula (I) or formula (Ia) or formula (II) or formula (IIa), can be used to treat certain leukemias, including FLT3-mediated leukemias.

In one embodiment, the present invention is a method of treating a patient suffering from an acute myeloid leukemia characterized by a FLT3 mutation. The method comprises administering to a patient a therapeutically effective amount of a compound of formula (I) or formula (Ia) or formula (II) or formula (IIa) or a pharmaceutically acceptable salt thereof.

As used herein, the term “leukemia” is a cancer of the blood or bone marrow characterized by an abnormal proliferation of blood cells, usually white blood cells (leukocytes). It is part of the broad group of diseases called hematological neoplasms.

Leukemias are selected from acute myeloid leukemia (AML), acute lymphocytic leukemia (ALL), chronic myeloid leukemia (CML) and chronic lymphocytic leukemia (CLL). In one embodiment, the present invention is a method of treating a patient suffering from a hairy cell leukemia (HCL).

Acute lymphocytic leukemia (also known as Acute Lymphoblastic Leukemia, or ALL) is the most common type of leukemia in young children. This disease also affects adults, especially those age 65 and older.

Acute myelogenous leukemia (also known as Acute Myeloid Leukemia, or AML) occurs more commonly in adults than in children. This type of leukemia was previously called acute nonlymphocytic leukemia.

Chronic lymphocytic leukemia (CLL) most often affects adults over the age of 55. It sometimes occurs in younger adults, but it almost never affects children.

Chronic myelogenous leukemia (CML) occurs mainly in adults. A very small number of children also develop this disease.

Hairy Cell Leukemia (HCL) leukemia is an incurable, indolent blood disorder in which mutated, partly matured B cells accumulate in the bone marrow. Its name is derived from the shape of the cells, which look like they are covered with short, fine, hair-shaped projections. Unlike any other leukemia, HCL is characterized by low white blood cell counts.

### 6. CSF-1R-Mediated Disorders

In one embodiment, the present invention is a method of treating a CSF-1R-mediated condition in a patient in need thereof, comprising administering to the patient a therapeutically effective amount of a compound of formula (I) or formula (la) or formula (II) or formula (IIa) or a pharmaceutically acceptable salt thereof.

Colony Stimulating Factor-1 Receptor (CSF-1R) signaling play an important role in the etiology of the disorders and conditions described above and is described, for example in Simoncic et al., Mol. Cel. Biol., Vol. 26, No. 11 (2006), pp. 41-49-4160; Yang et al., Ann. Rheum. Dis. (2006); 65, pp. 1671-1672; Irving et al., The FASEB J., Vol. 20 (2006): pp. E1315-E1326; Irving et al., The FASEB J., Vol. 20 (2006), pp. 1921-1923; Heinonen et al., PNAS (2006), vol. 103, no. 8, pp. 2776-2781; Conway et al., PNAS (2006), vol. 102, no. 44, pp. 16078-16083; Himes et al., The J. Immunol. (2006), 176: 2219-2228; Pixley et al., Trends in Cell Biol. (2004), Vol. 14, No. 11, pp. 628-638; U.S. Pat. App. Pub. No. 2006/0094081; U.S. Pat. App. Pub. No. 2006/0189623; U.S. Pat. App. Pub. No. 2006/0148812; U.S. Pat. App. Pub. No. 2006/0100201; U.S. Pat. No. 5,714,493; U.S. Pat. No. RE37,650. The relevant portions of all of these publications are incorporated herein by reference.

CSF-1R-mediated disorders include cardiovascular disease (e.g. artherial sclerosis), diseases with an inflammatory component including glomerulonephritis, prosthesis failure, sarcoidosis, congestive obstructive pulmonary disease, asthma, pancreatitis, HIV infection, psoriasis, diabetes, tumor related angiogenesis, age-related macular degeneration, diabetic retinopathy, restenosis, schizophrenia, skeletal pain caused by tumor metastasis or osteoarthritis, or visceral, inflammatory, and neurogenic pain, osteoporosis, Paget's disease, prosthesis failure, osteolytic sarcoma, myeloma, and tumor metastasis to bone, uterine cancer, stomach cancer, hairy cell leukemia, Sjogren's syndrom, or uveitis. The disorders include cancers such as osteolytic sarcoma, myeloma, and tumor metastasis to bone, uterine cancer, stomach cancer, hairy cell leukemia.

### Kinases Inhibited by the Compounds of the Invention

FLT3 (Fms-like tyrosine kinase; other names include CD135, FLK2 (Fetal liver kinase 2), STK1 (Stem cell kinase 1)) is a class III receptor tyrosine kinase (RTK) structurally related to the receptors for platelet derived growth factor (PDGF), colony stimulating factor 1 (CSF1), and KIT ligand (KL). These RTKs contain five immunoglobulin-like domains in the extracellular region and an intracelular tyrosine kinase domain split in two by a specific hydrophilic insertion (kinase insert). FLT3, closely related to PDGF receptors and c-Kit is, however, not inhibited by the small molecule inhibitors of PDGF and c-Kit; (G Del Zotto et al., J. Biol. Regulators Homeostatic Agents 15: 103-106, 2001).

The compounds of the present invention demonstrate inhibition of FLT3 at low nanomolar concentrations. In addition, the compounds of the present invention are highly specific for FLT3, and to a lesser extent V561D mutant PDGFRA (60-80 nM EC 50) and CSF-1R (200-400 nM EC50) within the broader class of tyrosine kinases. Inhibition of FLT3 is associated with modulation of transmigrating inflammatory cells by changing the antigen-presenting and signaling pathways that are mediated through dendritic cells and their recruitment, interaction and retraining of T-cells (Ajami et al. A. M., Boss, M. A. and Paterson, J. Compounds for treating autoimmune and demyelinating diseases. US Patent Appl. 2006/0189546A1, the disclosure of which is incorporated herein by reference).

In one embodiment, the present invention is a method of modulating the activity of FLT3 in a subject, comprising administering to the subject an effective amount of a compound of formula (I) or formula (Ia) or formula (II) or formula (IIa) or a pharmaceutically acceptable salt thereof.

The compounds of the present invention further demonstrate an unexpected and off target inhibitory effect on the MYLK2, light chain myosin kinase, a calcium dependent, serine-threonine kinase with no active site relationship to the Flt3 kinase class.

Although MYLK2 is structurally unrelated to Flt3, the inhibition of both kinases suggests a fortuitous and relevant functional connection in the mode of action of compounds in the present invention.

A related hallmark of invasive cells, shared by myeloid, lymphoid and cancer cells in their metastatic phase is the ability to breech cellular barriers, such as basement membranes, and therefore to invade and destroy tissues e.g. myelin, in the case of multiple sclerosis, or intestinal epithelial cells in Crohn's disease or ulcerative colitis (Behanna H A, Watterson D M and Ranaivo H R (2006) Biochim. Biophys. Acta 1763:1266-1274; Shen L, Black E D, Witkowski E D, Lencer W I, Guerriero V, Schneeberger E E and Turner J R (2006), J Cell. Sci. 119:2095-2106). Transmigration from blood vessels into synovium and bone in inflammatory arthritis serves as another case in point as does the recognized behavior of transmigrating tumor-associated macrophages and osteoclasts in metastatic cancers.

These so-called diapedesing cells have an overexpression of the molecular machinery for intracellular contractile force governed by the actomyosin regulatory complexes and the contraction cycle of the intracellular myosin complex. By the same token, the underlying barrier dysfunction, responsible for the leaky structures that permit destructive trafficking by invading immune system cells, is itself the result of an imbalance between intercellular adhesive forces, such as adherence junctions, and intracellular contractile forces that are linked to these adherence structures. These contractile forces also are mediated by actin-myosin cytoskeletal function. Activation of this cytoskeletal function requires phosphorylation of the myosin regulatory light chains (MLC) by myosin light chain kinase (MYLK2) (Kuhlmann C R W, Tamaki R, Gamerdinger M, Lessman V, Behl C, Kempski O S and Luhmann H J, J. Neurochem. 102:501-507).

Thus, it follows that inhibition of elevated levels of MYLK2 would prove beneficial in both suppressing the ability of activated immune cells to traffic and in suppressing the propensity of immunologically challenged barrier cells to pull themselves apart and exacerbate the impact of invasion into the underlying tissues. As such, the low nM inhibitory EC50 values reached with Compound (IV), an exemplar of the present invention, represents an unexpected and novel result, with significantly greater potency than the aryl sulfonamides known heretofore as MLCK (myosin light chain kinase) inhibitors (at micromolar EC50 levels) (Saitoh M, Ishikawa T, Matsushima S, Naka M, and Hidaka H (1987), J. Biol. Chem. 262(16):7796-7801).

MYLK2-mediated barrier dysfunction disorders include diseases with an inflammator component such as ulcerative colitis, Crohn's disease, bowel ischemia, colonic ileus, vasogenic ischemia, focal cerebral ischemia, hemorrhagic or septic shock, virus associated myelopathy, septic encephalopathy, glomerulonephritis, prosthesis failure, graft-versus-host disease, sarcoidosis, congestive obstructive pulmonary disease, asthma, pancreatitis, HIV infection, HIV-associated dementia, psoriasis, atopic dermatitis, diabetes, tumor related angiogenesis, restenosis, skeletal pain caused by tumor metastasis or osteoarthritis, or visceral, inflammatory, and neurogenic pain, osteolytic sarcoma, myeloma, and tumor metastasis to bone, uterine cancer, stomach cancer, hairy cell leukemia, Sjogren's syndrom, uveitis, uterine cancer, and stomach cancer.

In one embodiment, the present invention is a method of modulating the activity of MYLK2 in a subject, comprising administering to the subject an effective amount of a compound of formula (I) or formula (Ia) or formula (II) or formula (IIa) or a pharmaceutically acceptable salt thereof.

### Modes of Administration

The term “therapeutically effective amount” means an amount of the compound, which is effective in treating the named disorder or condition. When administered for the treatment or inhibition of a particular condition or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to treat the symptoms of the disease and its complications. The dosage to be used in the treatment of a specific individual typically must be subjectively determined by the attending physician. The variables involved include the specific condition and its state as well as the size, age and response pattern of the patient.

In certain embodiments, therapeutically effective amount means an amount sufficient to effect remyelination of nerve cells in a patient.

In treating a patient afflicted with a conditions described above, all of the disclosed compounds can be administered in any form or mode which makes the compound bioavailable in therapeutically effective amounts. For example, compounds of formula (I) or formula (Ia) or formula (II) or formula (IIa) can be administered in a form of a pharmaceutically acceptable salt. The term “pharmaceutically acceptable salt” means either an acid addition salt or a basic addition salt, whichever is possible to make with the compounds of the present invention. “Pharmaceutically acceptable acid addition salt” is any non-toxic organic or inorganic acid addition salt of the base compounds represented by formula (I) or formula (Ia). Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points. “Pharmaceutically acceptable basic addition salts” means non-toxic organic or inorganic basic addition salts of the compounds of formula (I) or formula (Ia) or formula (II) or formula (IIa). Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

Compounds of the present invention can be administered by a number of routes including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The disclosed compounds are administered by any suitable route, including, for example, orally in capsules, suspensions or tablets.

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The compound of formula (I) or formula (Ia) or formula (II) or formula (IIa) of this invention may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

In the embodiments in which the compounds of the invention are used to treat cancer, the dosage range at which the disclosed compounds, for example, compounds of formula (I) or formula (Ia) or formula (II) or formula (IIa), including the above-mentioned examples thereof, exhibit their ability to act therapeutically can vary depending upon the severity of the condition, the patient, the formulation, other underlying disease states that the patient is suffering from, and other medications that may be concurrently administered to the patient. Generally, the compounds described herein will exhibit their therapeutic activities at dosages of between about 0.1 mg/m² free base equivalent per square meter of body surface area/single dose to about 1000 mg/m² free base equivalent per square meter of body surface area/single dose. These dosages can be administered, for example, once per week, once every other week, once every third week or once per week for three out of every four weeks.

Alternatively, when used to treat cancer, the disclosed compounds can be administered daily (typically orally). Daily dose of administration of the compounds of the present invention can be repeated, in one embodiment, for one week. In other embodiments, daily dose can be repeated for one month to six months; for six months to one year; for one year to five years; and for five years to ten years. Representative total daily doses would include those in the range of 10-2000 mg. The total daily dose can be divided into equal doses and administered twice daily, thrice daily, or four times daily. In other embodiments, the length of the treatment by repeated administration is determined by a physician.

When used to treat other indications, the dosage range at which the disclosed compounds of formula (I) or formula (Ia) or formula (II) formula (IIa) exhibit their ability to act therapeutically can vary depending upon the severity of the condition, the patient, the formulation, other underlying disease states that the patient is suffering from, and other medications that may be concurrently administered to the patient. Generally, the inventive compounds of the invention will exhibit their therapeutic activities at dosages of between about 0.001 mg/kg of patient body weight/day to about 100 mg/kg of patient body weight/day. For example, the dosage can be 0.1-100 mg/kg per every other day or per week.

### Combination Therapies

The compounds used in the present invention can be administered alone or in combination with one or more other pharmaceutically active agents that are effective against the inflammatory condition and/or the demyelinating disorder being treated.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

In one embodiment, the compounds of the present invention are administered in combination with an anti-inflammatory agent. The anti-inflammatory agent can be adrenocorticotropic hormone, a corticosteroid, an interferon, glatiramer acetate, or a non-steroidal anti-inflammatory drug (NSAID).

Examples of suitable anti-inflammatory agents include corticosteroid such as prednisone, methylprednisolone, dexamethasone cortisol, cortisone, fludrocortisone, prednisolone, 6α-methylprednisolone, triamcinolone, or betamethasone.

Other examples of suitable anti-inflammatory agents include NSAIDs such as aminoarylcarboxylic acid derivatives (e.g., Enfenamic Acid, Etofenamate, Flufenamic Acid, Isonixin, Meclofenamic Acid, Niflumic Acid, Talniflumate, Terofenamate and Tolfenamic Acid), arylacetic acid derivatives (e.g., Acematicin, Alclofenac, Amfenac, Bufexamac, Caprofen, Cinmetacin, Clopirac, Diclofenac, Diclofenac Sodium, Etodolac, Felbinac, Fenclofenac, Fenclorac, Fenclozic Acid, Fenoprofen, Fentiazac, Flubiprofen, Glucametacin, Ibufenac, Ibuprofen, Indomethacin, Isofezolac, Isoxepac, Ketoprofen, Lonazolac, Metiazinic Acid, Naproxen, Oxametacine, Proglumrtacin, Sulindac, Tenidap, Tiramide, Tolectin, Tolmetin, Zomax and Zomepirac), arylbutyric acid ferivatives (e.g., Bumadizon, Butibufen, Fenbufen and Xenbucin) arylcarboxylic acids (e.g., Clidanac, Ketorolac and Tinoridine), arylproprionic acid derivatives (e.g., Alminoprofen, Benoxaprofen, Bucloxic Acid, Carprofen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Piketoprofen, Piroprofen, Pranoprofen, Protinizinic Acid, Suprofen and Tiaprofenic Acid), pyrazoles (e.g., Difenamizole and Epirizole), pyrazolones (e.g., Apazone, Benzpiperylon, Feprazone, Mofebutazone, Morazone, Oxyphenbutazone, Phenylbutazone, Pipebuzone, Propyphenazone, Ramifenazone, Suxibuzone and Thiazolinobutazone), salicyclic acid derivatives (e.g., Acetaminosalol, 5-Aminosalicylic Acid, Aspirin, Benorylate, Biphenyl Aspirin, Bromosaligenin, Calcium Acetylsalicylate, Diflunisal, Etersalate, Fendosal, Flufenisal, Gentisic Acid, Glycol Salicylate, Imidazole Salicylate, Lysine Acetylsalicylate, Mesalamine, Morpholine Salicylate, 1-Naphthyl Sallicylate, Olsalazine, Parsalmide, Phenyl Acetylsalicylate, Phenyl Salicylate, 2-Phosphonoxybenzoic Acid, Salacetamide, Salicylamide O-Acetic Acid, Salicylic Acid, Salicyloyl Salicylic Acid, Salicylsulfuric Acid, Salsalate and Sulfasalazine), thiazinecarboxamides (e.g., Droxicam, Isoxicam, Piroxicam and Tenoxicam), ε-Acetamidocaproic Acid, S-Adenosylmethionine, 3-Amino-4-hydroxybutyric Acid, Amixetrine, Bendazac, Benzydamine, Bucolome, Difenpiramide, Ditazol, Emorfazone; Guaiazulene, Ketorolac, Meclofenamic Acid, Mefenamic Acid, Nabumetone, Nimesulide, Orgotein, Oxaceprol, Paranyline, Perisoxal, Pifoxime, Piroxicam, Proquazone, Tenidap and a COX-2 inhibitor (e.g., Rofecoxib, Valdecoxib and Celecoxib).

Further examples of anti-inflammatory agents include aspirin, a sodium salicylate, choline magnesium trisalicylate, salsalate, diflunisal, sulfasalazine, olsalazine, a para-aminophenol derivatives, an indole, an indene acetic acid, a heteroaryl acetic acid, an anthranilic acid, an enolic acid, an alkanones, a diaryl-substituted furanone, a diaryl-substituted pyrazoles, an indole acetic acids, or a sulfonanilide.

In some embodiments, the compounds of the present invention can be administered in combination with immunotherapeutic agents such as interferons and anti-integrin blocking antibodies like natalizumab.

Examples of agents suitable for treating demyelinating disorders include Pirfenidone, Epalrestat, Nefazodone hydrochloride, Memantine hydrochloride, Mitoxantrone hydrochloride, Mitozantrone hydrochloride, Thalidomide, Roquinimex, Venlafaxine hydrochloride, Intaxel, Paclitaxel, recombinant human nerve growth factor; nerve growth factor, ibudilast, Cladribine, Beraprost sodium, Levacecarnine hydrochloride; Acetyl-L-carnitine hydrochloride: Levocarnitine acetyl hydrochloride, Droxidopa, interferon alfa, natural interferon alpha, human lymphoblastoid interferon, interferon beta-1b, interferon beta-Ser, Alemtuzumab, Mycophenolate mofetil, Zoledronic acid monohydrate, Adapalene, Eliprodil, Donepezil hydrochloride, Dexanabinol, Dexanabinone, Xaliproden hydrochloride, interferon alfa-n3, lipoic acid, thioctic acid, Teriflunomide, Atorvastatin, Pymadin, 4-Aminopyridine, Fampridine, Fidarestat, Priliximab, Pixantrone maleate, Dacliximab, Daclizumab, Glatiramer acetate, Rituximab, Fingolimod hydrochloride, interferon beta-1a, Natalizumab, Abatacept, Temsirolimus, Lenercept, Ruboxistaurin mesilate hydrate, Dextromethorphan/quinidine sulfate, Capsaicin, Dimethylfumarate or Dronabinol/cannabidiol.

In some embodiments, the compounds of the present invention can be administered in combination with one or more other pharmaceutically active agents that are effective against multiple sclerosis. Examples of such agents include the interferons (interferon beta 1-a, beta 1-b, and alpha), glatiramer acetate or corticosteroids such as methylprednisolone and prednisone as well as chemotherapeutic agents such as mitoxantrone, methotrexate, azathioprine, cladribine cyclophosphamide, cyclosporine and tysabri.

Further examples of pharmaceutically active agents that are effective against multiple sclerosis and are suitable to be administered in combination with compounds of the present invention include compounds of the following structural formulae:

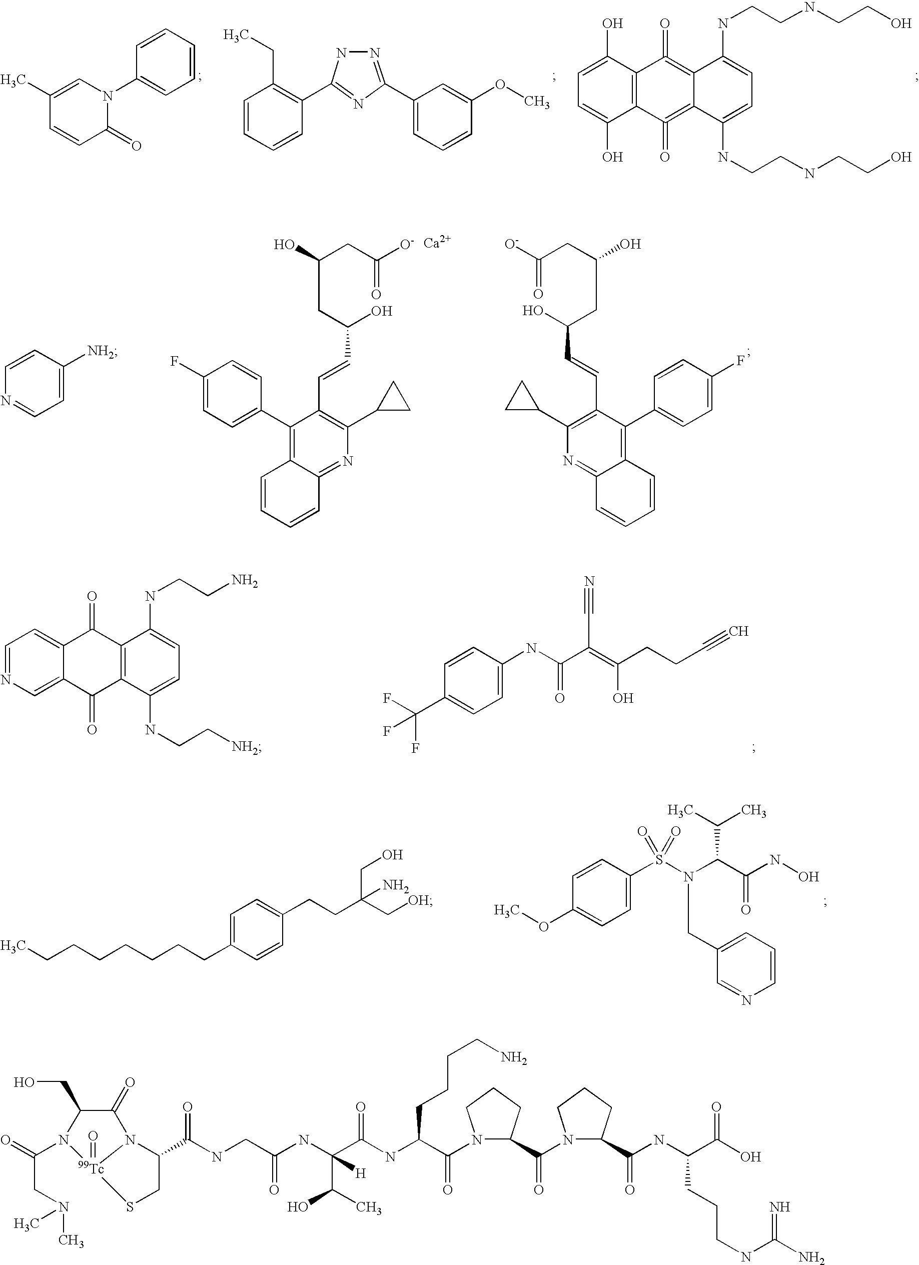

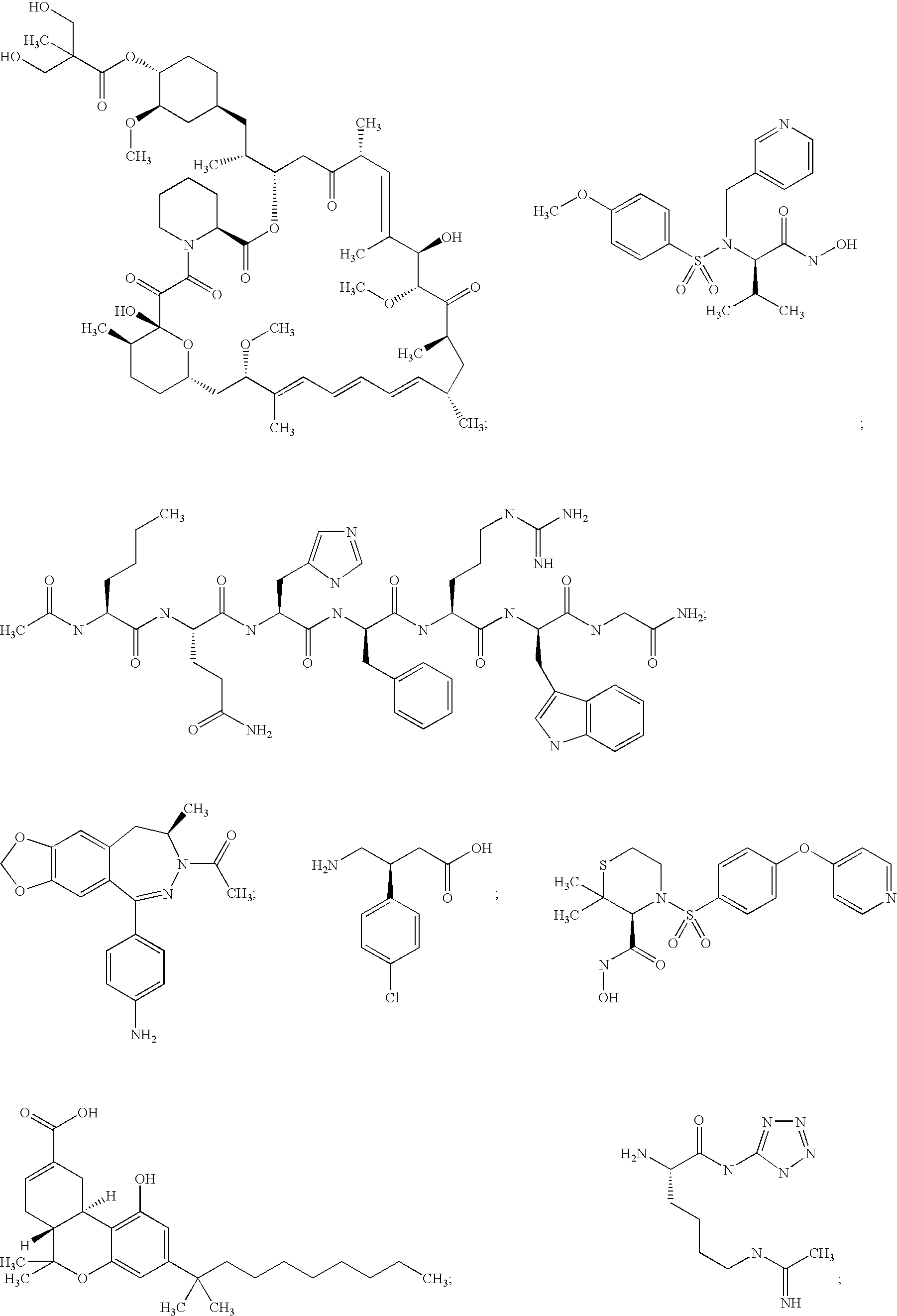

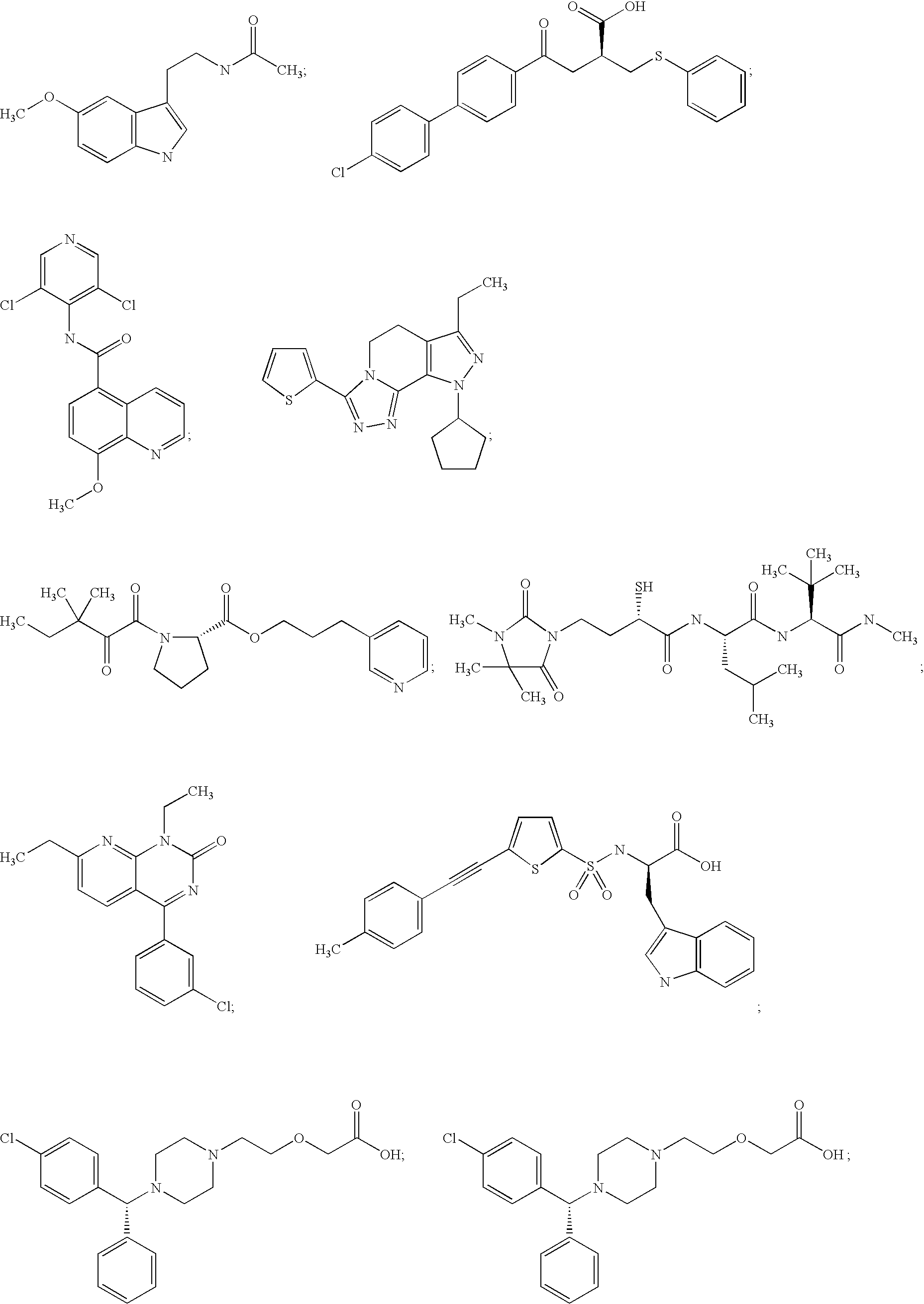

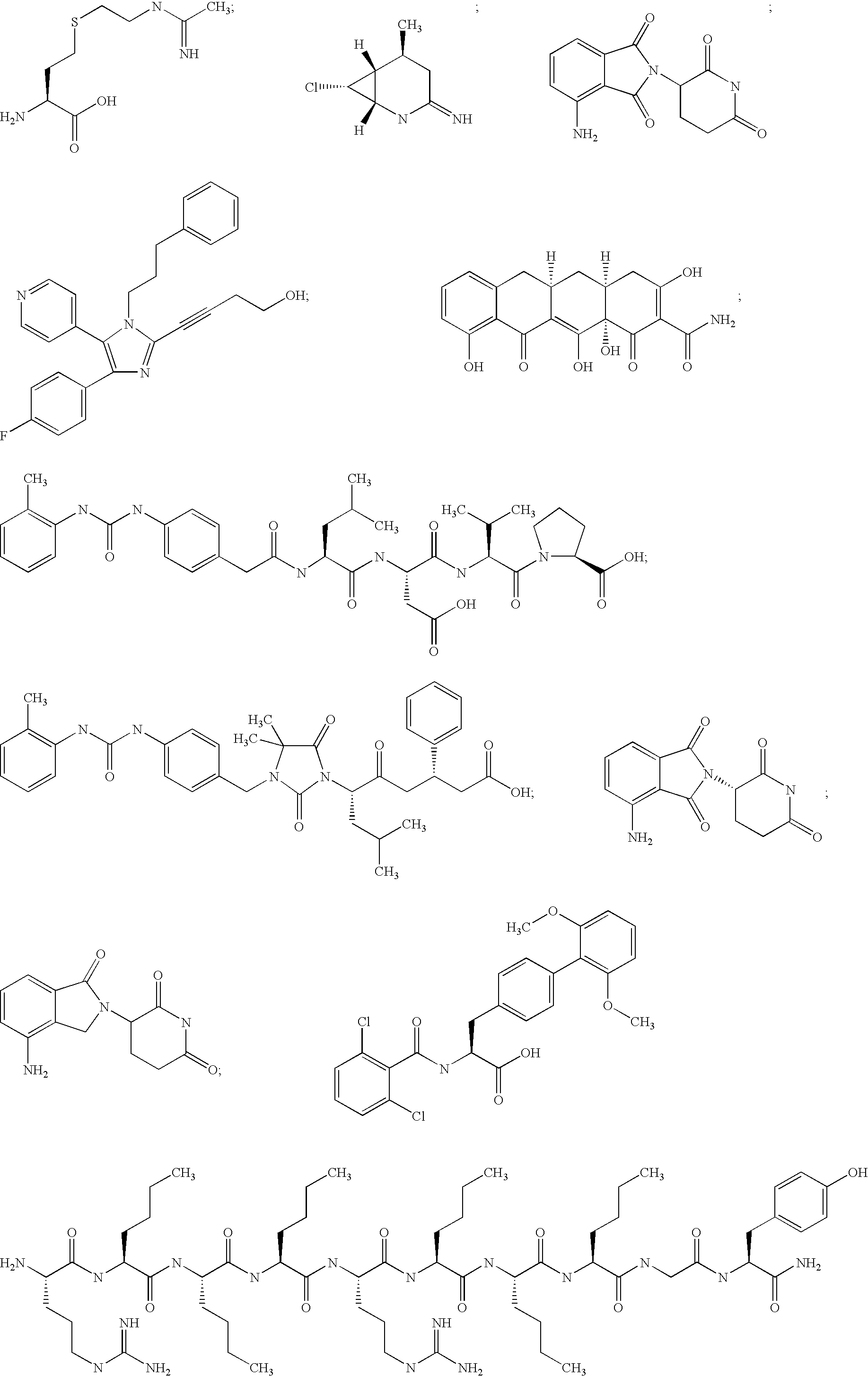

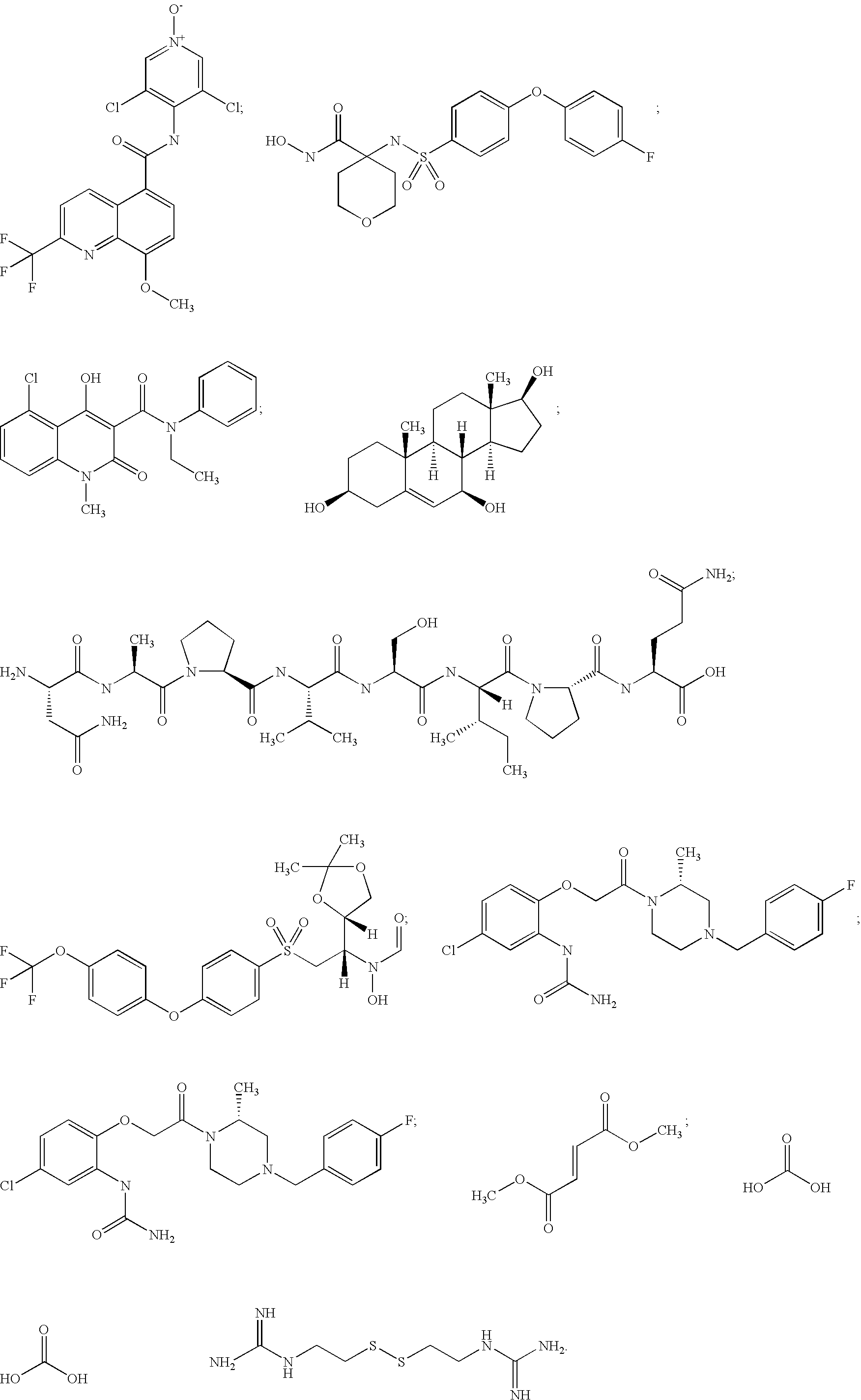

Further examples of pharmaceutical agents that can be co-administered with the compounds of formula (I) or formula (Ia) or formula (II) or formula (IIa) include:

T-cell receptor (TCR) Vβ6 CDR2 peptide vaccine consisting of TCR Vβ6, amino acid sequence 39-58, Leu-Gly-Gln-Gly-Pro-Glu-Phe-Leu-Thr-Tyr-Phe-Gln-Asn-Glu-Ala-Gln-Leu-Glu-Lys-Ser (SEQ ID NO:1);

Myelin basic protein immunogen peptide, aminoacid sequence 75-95. Lys-Ser-His-Gly-Arg-Thr-Gln-Asp-Glu-Asn-Pro-Val-Val-His-Phe-Phe-Lys-Asn-Ile-Val-Thr (SEQ ID NO:2);

Tiplimotide, myelin basic protein immunogen vaccine peptide, aminoacid sequence 83-99, D-Ala-lys-pro-val-val-his-leu-phe-ala-asp-ile-val-thr-pro-arg-thr-pro, (SEQ ID NO:3);

Myelin basic protein immunogen peptide, aminoacid sequence 82-98, Asp-glu-asp-pro-val-val-his-phe-phe-lys-asp-ile-val-thr-pro-arg-thr, (SEQ ID NO:4);

Adrenocorticotropic hormone (ACTH), Ser-Tyr-Ser-met-glu-his-phe -arg-try-gly-lys-pro-val-gly-lys-lys-arg-arg-pro-val-lys-val-tyr-pro-asp-gly-ala-glu-asp-glu-leu-ala-glu-ala-phe-pro-leu-glut-phe, (SEQ ID NO:5).

Further examples of pharmaceutically active agents that are effective against multiple sclerosis and are suitable to be administered in combination with compounds of the present invention include:

3-4 diaminopyridine; ABT-874; Actos® (pioglitazone); ALCAR (acetyl-L-carnitine); Alpha lipoic acid; AndroGel® (testosterone gel); combination of trimethoprim and vitamin C; combination of azithromycin and rifampin; minocycline; donezepil HCL; Avandia® (rosiglitazone maleate; combination of IFN beta-1a) and acetaminophen, ibuprofen or prednisone; combination of Avonex® (interferon beta-1a)+CellCept® (mycophenolate mofetil); combination of Avonex® (interferon beta-1a) and Copaxone® (glatiramer acetate); combination of Avonex® (interferon beta-1a) and doxycycline; combination of Avonex® (interferon beta-1a) and EMLA (lidocaine and prilocaine) anesthetic cream; Avonex® (interferon beta-1a) and estrogen and progesterone; combination of Avonex® (interferon beta-1a)+Fludara® (fludarabine phosphate); combination of Avonex® (interferon beta-1a) and methotrexate and leucovorin rescue; combination of Avonex® (interferon beta-1a) and methotrexate and methylprednisolone; combination of Avonex® (interferon beta-1a) and Novantrone® (mitoxantrone); combination of Avonex® (interferon beta-1a) and Prozac® (fluoxetine); combination of Avonex® (interferon beta-1a) and Topamax® (topiramate); combination of Avonex® (interferon beta-1a) and Zocor® (simvastatin); AVP-923 (dextromethorphan/quinidine); combination of Betaseron® (interferon beta-1b) and Imuran® (azathioprine); combination of Betaseron® (interferon beta-1b) and Copaxone® (glatiramer acetate); combination of BHT-3009-01 and Lipitor® (atorvastatin); Bone marrow/peripheral stem cell transplant; CellCept® (mycophenolate mofetil); combination of CellCept® (mycophenolate mofetil) and Avonex® (interferon beta-1a); Oral cladribine; CNTO 1275 (monoclonal antibody); combination of Copaxone® (glatiramer acetate) and Antibiotic therapy (minocycline); combination of Copaxone® (glatiramer acetate) and Novantrone® (mitoxantrone); combination of Copaxone® (glatiramer acetate) and prednisone; combination of Copaxone® (glatiramer acetate) and Proventil® (albuterol); Cyclophosphamide; Daclizumab; Deskar® (pirfenidone); Estriol; Fumaric acid esters; Gabitril® (tiagabine HCL); Ginkgo biloba; IDEC-131 (anti-CD40L or anti-CD 154); the combination of Immunoglobulin and methylprednisolone; Inosine; Interferon tau; Lamictal® (lamotrigine); Lexapro® (escitalopram); Lipitor® (atorvastatin); combination of Lipitor® (atorvastatin) and Rebif® (interferon beta-1a); combination of Lymphocytapheresis (removal of immune cells), Imuran® (azathioprine) and prednisone; MBP8298; Methylprednisolone; combination of Methylprednisolone and Avonex (interferon beta-1a); Modiodal (modafinil); NBI-5788 (altered peptide ligand); combination of Novantrone® (mitoxantrone for injection concentrate) and Avonex® (Interferon beta-1a) or Copaxone® (glatiramer acetate); Omega-3 Fatty Acid Supplementation; Pixantrone (BBR 2778); combination of Provigil® (modafinil) and Avonex® (interferon beta-1a); Rapamune® (sirolimus); RG2077; Rituxan® (rituximab); Rolipram (phosphodiesterase-4 inhibitor); SAIK-MS (laquinimod, ABR-215062); T cell vaccination; Teriflunomide; Tetrahydrocannabinol; Tetrahydrocannabinol (dronabinol); Thalamic stimulation; combination of Tysabri® (natalizumab) and Avonex® (interferon beta-1a); combination of Tysabri® (natalizumab) and Copaxone® (glatiramer acetate); and Viagra® (sildafenil citrate).

Further example of pharmaceutically active agents that are effective against multiple sclerosis and are suitable to be administered in combination with compounds of the present invention is Copaxone (Glatiramer), which can be orally co-administered with the compounds of the present invention.

In other embodiments, pharmaceutically active agents that are effective against multiple sclerosis and are suitable to be administered in combination with compounds of the present invention include compounds include: Mylinax, an oral formulation of cladrlbine used in leukaemia treatment, developed by Serono/Ivex; Teriflunomide, a metabolite of Arava, an oral immunosuppressant, developed by Sanofi-Aventis; FTY 720, an oral immunomodulator (Sphingosine-1-phosphate receptor agonist), developed by Novartis; MBP 8298, a synthetic myelin basis protein designed to reduce the emergence of antibodies directed against the myelin, developed by Bio MS Medical; an orphan drug 4-aminopyridline (4-AP), a potassium channel blocker, developed by Acorda; Gamunex, an intravenous immunoglobulin formulation, developed by Bayer; BG-12 fumarate, a second generation oral futnarate, developed by Biogen Idec/Fumapharm; Temsirolimus, a T-lymphocytes proliferation blocker, developed by Wyeth; E-2007, an AMPA receptor agonist, developed by Eisal; Campath, a humanized antibody directed against CD52, developed by Genzyme; Neuro Vax, a vaccine, developed by Immune Response; Zocor, a statin, developed by Merck; NBI 5788, a myelin-mimicking peptide ligand, developed by Neurocrine; Tauferon, Interferon tau, developed by Pepgen; Zenapax, a humanized anti-CD25 immunosuppressive antibody, developed by Protein Design; a combination of MS-IET and EMZ 701, a methyl donator, developed by Transition Therapeutics; Laquinlmod, an oral formulation of a derivative of linomide, developed by Active Biotech/Teva; deskar pirfenidone, a INF-alpha inhibitor, developed by Mamac; ATL-1102, a second generation antisense inhibitor targeting VLA4, developed by Antisense Therapeutics.

In some embodiments, compounds of formula (A) can be administered in combination with antivascular agents, in particular agents inhibiting the growth factor receptors, Epidermal Growth Factor Receptor (EGFR), Vascular Epidermal Growth Factor Receptor (VEGFR), and Fibroblast Growth Factor Receptor (FGFR). Examples of such agents include, Iressa, Tarceva, Erbitux, Pelitinib, AEE-788, CP-547632, CP-547623, Tykerb (GW-2016), INCB-7839, ARRY-334543, BMS-599626, BIBW-2992, Falnidamol, AG1517, E-7080, KRN-951, GFKI-258, BAY-579352, CP-7055, CEP-5214, Sutent, Macugen, Nexavar, Neovastat, Vatalanib succinate, GW-78603413, Lucentis, Teavigo, AG-13958, AMG-706, Axitinib, ABT-869, Evizon, Aplidin, NM-3, PI-88, Coprexa, AZD-2171, XL-189, XL-880, XL-820, XL-647, ZK-CDK, VEGFTrap, OSI-930, Avastin, Revlimid, Endostar, Linomide, Xinlay, SU-668, BIBF-1120, BMS-5826624, BMS-540215.

In some embodiments, compounds of formula (I) or formula (Ia) or formula (II) or formula (IIa) can be administered in combination with agents that affect T-cell homing, extravastion and transmigration. Examples of such agents include, FTY-720PKI-166, PTK-787, SU-11248.

In some embodiments, compounds of formula (I) or formula (Ia) or formula (II) or formula (IIa) can be administered in combination with agents inhibiting VLA-4. Examples of such agents include, Tysabri, Bio-1211, HMR-1031, SB-683698, RBx-4638, RO-0272441, RBx-7796,SB-683699, DW-908e, AJM-300, and PS-460644.

In certain embodiments, the compound of formula (I) or formula (Ia) or formula (II) or formula (IIa) can be administered alone or in combination with an anti-cancer agent.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

Anti-cancer agents that can be employed in combination with the compounds of the invention include Taxol™ (also referred to as “paclitaxel”, and compounds that have the basic taxane skeleton), Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin;. cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine: epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate: liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin and zorubicin hydrochloride.

Other anti-cancer drugs that can be employed in combination with the compounds described herein include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; amsacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; tluasterone; fludarabine; tluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; pertlubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors: protein A-based immune modulator; protein kinase C inhibitor; protein kinase C inhibitors, microalgal; protein tyrosine phosphatase inhibitors; purine nucleoside phosphorylase inhibitors; purpurins: pyrazoloacridine; pyridoxylated hemoglobin polyoxyethylene conjugate: raf antagonists: raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tel telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred anti-cancer drugs are 5-fluorouracil and leucovorin.

Other chemotherapeutic agents that can be employed in combination with the compounds of the invention include but are not limited to alkylating agents, antimetabolites, natural products, or hormones.

The invention is illustrated by the following examples, which are not intended to be limiting in any way. The skilled practioner will be able to exercise routine judgment for the selection of suitable starting materials in order to prepare specific products. the order of synthetic steps. and the need for protecting groups for remote functionalities.

### Exemplification

### Example 1

### Synthesis of the Compounds of the Invention

Compounds of formula (I) or formula (Ia) and formula (II) or formula (IIa) can be synthesized according to the following examples. It is understood by those skilled in the art of organic synthesis that the substitution patterns of the starting materials determines the substitution patterns of the products, and the skilled practioner will be able to exercise routine judgment for the selection of suitable starting materials in order to prepare specific products, the order of synthetic steps, and the need for protecting groups for remote functionalities.

The chemistry of protecting groups can he found, for example, in Greene, et al., Protective Groups in Organic Synthesis, 2d. Ed., Wiley & Sons. 1991, the entire disclosure of which is herein incorporated by reference.

Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but one skilled in the art can determine such conditions by routine optimization procedures.

### (A) 2-Fluoro-5-nitro-6-(1H-indazol-5-ylamino)benzoic acid

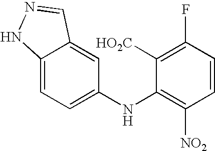

The starting material 3-nitro-2,6-difluorobenzoic acid was prepared by nitration of 2,6-difluorobenzoic acid with potassium nitrate in sulfuric acid as described in part by Kuramoto Y, Ohshita Y, Yoshida J, Yazaki A, Shiro M. and Koike T, J. Med. Chem. (2003), 46:1905-1917) and isolated as described in a similar preparation by Yoshida Y, Barrett D, Azami H, Morinaga C, Matsumoto S, Matsumoto Y, and Takasugi H, (1999), Bioorg. Med. Chem. 7:2647-2666. 3-Nitro-2,6-difluorobenzoic acid (20.0 g, 98.44 mmol) was added to a solution of ethanol (100 mL) and water (100 mL). The acid solution was cooled to 10° C. and triethylamine (25.09 mL) added by drops under rapid stirring to ensure the temperature did not exceed 40° C. 5-Aminoindazole (13.01 g, 98.44 mmol) was then added in portions and the combined mixture heated to 70° C. for 16 hours. A solution of water (100 mL) and concentrated HCl (100 mL) was prepared, heated to 60° C. and placed under vigorous stirring. The reaction mixture, still at 70° C., was transferred to the HCl/water solution in small portions and allowed to cool to room temperature. The mixture was stirred for a further 4 hours to ensure maximum precipitation. The resulting precipitate was filtered off and washed with water (2×60 mL) and dried in a vacuum oven overnight (28.3 g, 89.56 mmol, 94%). ¹H δ (d6-DMSO): 6.97 (1H, dd, ArH, J=8.8, 2.4 Hz), 7.18 (1H, d, ArH, J=2.4 Hz), 7.43 (1H, d, ArH, J=8.8 Hz), 7.45 (1H, d, ArH, J=8.8 Hz), 8.10 (1H, d, ArH, J=8.8 Hz), 8.99 (1H, s, ArH), 9.15 (1H, s, NH), 9.87 (1H, s, NH); HPLC: Rₜ=3.52 min.; LRMS: m/z=315.4 (M−H)

### (B) 1-Fluoro-4-nitropyrazolo[7,8a]-acridin-9(10H)-one

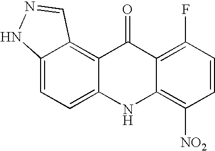

A suspension of 2-fluoro-5-nitro-6-(1H-indazol-5-ylamino)benzoic acid (20.0 g, 65.096 mmol) in chloroform under argon had freshly distilled POCl₃ (24.3 mL, 260.383 mmol) added to it. The mixture was heated to 80° C. for 16 hours. Once cooled, ethanol (50 mL) was added slowly to quench the excess POCl₃ reaction and then stirred for 30 minutes at room temperature. At this point, the mixture was reduced to dryness under vacuum. Water (100 mL) was added to the residue and saturated sodium bicarbonate solution added until the pH=8. The mixture was then stirred for 60 minutes at room temperature and the precipitate filtered off, washed with water (2×80 mL) and dried overnight in a vacuum oven (19.219 g, 64.445 mmol, 99%). ¹H δ (d6-DMSO): NMR unavailable since compound very insoluble; HPLC: Rₜ=6.22 min.; LRMS: m/z=299.1 (M+H)

### (C) 1-Fluoro-4-nitropyrazolo[7,8a]-imidazo[4,5,1-de]-acridin-9(10H)-one (Compound XVIII)

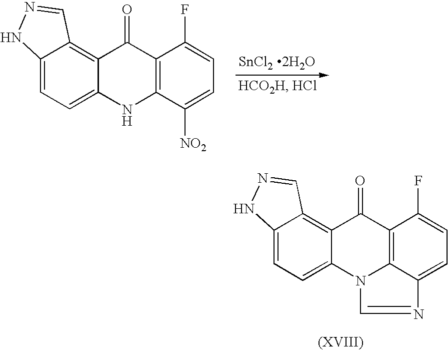

To a slurry of starting material (7.112 g, 23.846 mmol) in formic acid (75 mL), was added SnCl₂.2H₂O (23.07 g, 102.25 mmol) in concentrated HCl (15 mL). The combined mixture was stirred at room temperature for 30 minutes then heated to 95° C. for 20 hours. Once cooled, the solid was filtered off and partially dried before being slurried in saturated sodium bicarbonate solution and stirred for 60 mins. The precipitate was then filtered off and washed with water (2×100 mL) and dried in a vacuum oven overnight (6.303 g, 22.654 mmol, 95%).

### (D) 4-Amino-1-fluoropyrazolo[7,8a]-acridin-9(10H)-one

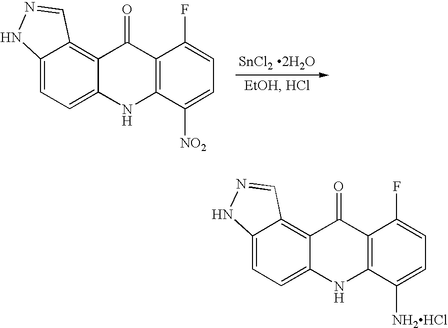

1-Fluoro-4-nitropyrazolo[7,8a]-acridin-9(10H)-one (10 g, 33.5 mmol) in ethanol (100 mL) had a solution of tin chloride dihydrate (22.7 g, 100.5 mmol) in concentrated HCL (30 mL). The mixture was heated to 80° C. for 25 hours until the t.l.c. showed no starting material remained. The mixture was cooled, the solid filtered off and washed with water (4×100 mL) then dried under vacuum. ¹H δ (d6-DMSO): 5.60-6.20 (4H, br, NH, HCl), 6.96 (1H, dd, ArH, J=8.8, 8.4 Hz), 7.50 (1H, d, ArH, J=4.4 Hz), 7.90 (1H, d, ArH, J=9.2 Hz), 8.03 (1H, d, ArH, J=9.2 Hz), 8.81 (1H, s, ArH), 11.89 (1H, s, NH); HPLC: Rₜ=3.32 min.; LRMS: m/z=277.2 (M−H).

### (E) 1-Fluoro-4-nitromazolo[7,8a]-imidazo[4,5,1-de]-acridin-9(10H)-one (Compound XVIII)

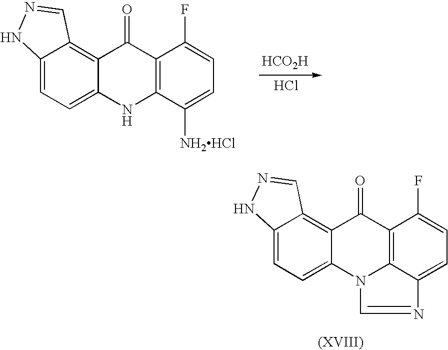

4-Amino-1-fluoropyrazolo[7,8a]-acridin-9(10H)-one (8 g. 29.8 mmol) in formic acid (100 mL) and concentrated HCl (20 mL) was heated to 95° C. for 35 hours. The mixture was cooled and the solid filtered off, washed with water (4×100 mL) and dried under vacuum. No further purification was carried out.

### (F) Compound (V)

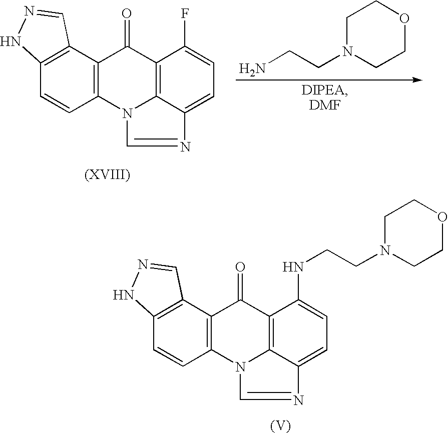

Compound XVIII (0.294 g, 1.057 mmol), 2-morpholine ethylamine (0.220 g, 1.961 mmol, 0.222 mL), diisopropylethylamine (0.273 g, 2.114 mmol, 0.368 mL) and dimethylacetamide (2.0 mL) were combined in a 5 mL reaction tube and heated to 150° C. under microwave irradiation for 20 minutes. Once cooled, the solvent was removed and the residue columned over silica gel (0-20% MeOH in CHCl₃). The desired fractions were pooled and the solvent removed to yield the target compound. ¹H δ (d6-DMSO): 2.41 (2H, m, CH₂NCH₂), 2.68 (2H, m, CH₂), 3.19 (2H, m, CH₂NCH₂), 3.36 (2H, m, NHCH₂), 3.63 (4H, m, CH₂OCH₂), 6.89 (1 H, d, ArH, J=8.6 Hz), 8.01 (1H, d, ArH, J=8.6 Hz), 8.16 (1H, d, ArH, J=9.0), 8.44 (1H, d, ArH, J=9.0 Hz), 9.00 (1H, s, Pyrazole-H), 9.07 (1H, m, NH), 9.29 (1H, s, Imidazole-H), 13.69 (1H, s, N═NH); HPLC: Rₜ=3.55 min.; LRMS: 389.0 (M+1).

### (G) 1-(3-Dimethylamino-propylamino)-4-nitropyrazolo[7,8a]-acridin-9(10H)-one

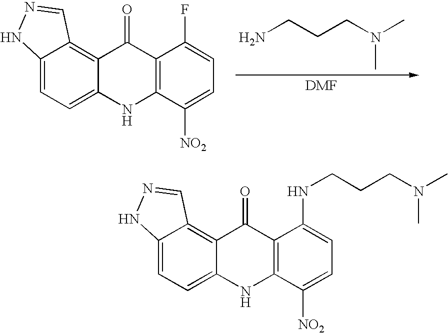

To a stirred slurry of 1-fluoro-4-nitropyrazolo[7,8a]-acridin-9(10H)-one (8.75 g, 29.34 mmol) in DMF (220 mL) was added N,N-dimethyl-1,3-propanediamine (8.99 g, 88.02 mmol, 3 equiv). The mixture was stirred at room temperature for 18 hours and then concentrated to dryness. The residue was treated with aqueous sodium bicarbonate solution (400 mL) and stirred for 15 minutes. The precipitate was collected by filtration, rinsed with water and ethyl acetate. The filter cake was vacuum-dried to afford the title compound (8.11 g, 72% yield) as a yellow solid. ¹H δ (400 MHz, DMSO-d₆): 13.60 (1H, broad s), 12.68 (1H, s), 12.13 (1H, t, J=6.5 Hz), 8.78 (1H, s), 8.35 (1H, d, J=9.8 Hz), 7.97 (1H, m), 7.91 (1H, m), 6.58 (1H, d, J=9.8 Hz), 3.49 (2H, m), 2.37 (2H, t, J=6.7 Hz), 2.18 (6H, s), 1.83 (2H, m); LRMS: m/z=381.4 (M+H).

### (H) Compound (IV)

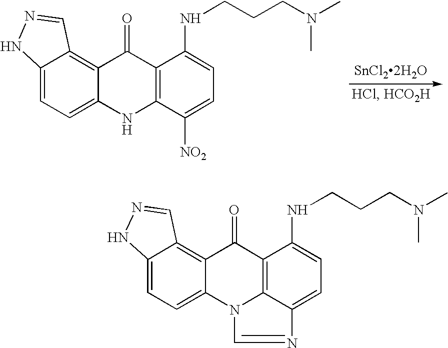

To a stirred solution of 1-(3-dimethylamino-propylamino)-4-nitropyrazolo[7,8a]-acridin-9(10H)-one (8.05 g, 21.10 mmol) in formic acid (250 mL) was added a solution of tin (II) chloride dihydrate (21.43 g, 94.95 mmol) in 37% hydrochloric acid (28 mL) at room temperature. The mixture was heated at a gentle reflux for 22 hours and then concentrated to dryness. The resulting solid was dissolved in water, basified with an excess of aqueous sodium bicarbonate (1.2 L), and stirred 30 minutes for granulation. The precipitate was collected by filtration, rinsed with water, and vacuum-dried to give the crude product which contained inorganic impurities (tin hydroxides). To remove the inorganic impurities, the crude product was filtered through a silica gel pad, eluting with DMF, affording the title compound (3.12 g, 41% yield) as an orange-yellow solid.

Representative compounds with the fused pyrazole scaffold are prepared in a similar manner. For example compounds (III) and (IV) were prepared by the addition of 5-N,N-dimethylamino pentylamine and 3-N,N-dimethylamino propylamine to compound (VI), following the method used for (V), namely, by treating compound (XVIII) with dialkylamino-alkyl amines using the same reaction stoichiometry shown in the model reaction with 2-morpholine ethylamine.

The resulting compounds conformed to theory as evidenced by their NMR and mass spectroscopic features, shown in the Table 1 that provides the analytical properties for compound (XVIII), and its derivatives.

TABLE 1
Analytical characterization of representative compounds

| | | | LRMS | HPLC |
|---|---|---|---|---|
| | Structure | ¹H NMR (δ) | (m/z) | (Rₜ) |
| (XVIII) | 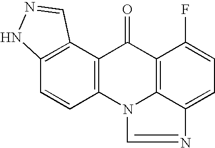 | 7.45 (1H, dd, ArH, J = 8.8, 8.8 Hz), 8.20 (1H, d, ArH, J = 9.2 Hz), 8.24 (1H, dd, ArH, J = 8.8, 3.4), 8.44 (1H, d, ArH, J = 9.2 Hz), 8.96 (1H, s, ArH), 9.64 (1H, s, ArH), 13.77 (1H, s, NH) | 279.0 (M + H) | 5.27 min. |
| (V) | 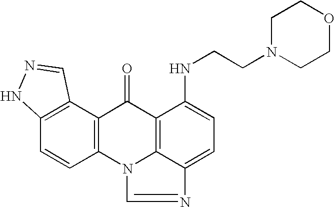 | 2.41 (2H, m, CH₂NCH₂), 2.68 (2H, m, CH₂), 3.19 (2H, m, CH₂NCH₂), 3.36 (2H, m, NHCH₂), 3.63 (4H, m, CH₂O CH₂), 6.89 (1H, d, ArH, J = 8.6 Hz), 8.01 (1H, d, ArH, J = 8.6 Hz), 8.16 (1H, d, ArH, J = 9.0 Hz), 8.44 (1H, d, ArH, J = 9.0 Hz), 9.00 (1H, s, ArH), 9.07 (1H, m, NH), 9.29 (1H, s, ArH), 13.69 (1H, s, N═NH) | 389.0 (M + H) | 3.52 min. |
| (IV) | 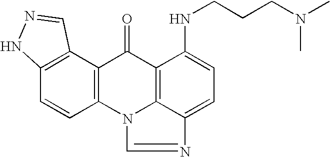 | 2.09 (2H, t, CH₂, J = 7.2 Hz), 2.75 (6H, s, N(CH₃)₂, 3.18 (2H, t, NCH₂, J = 7.2 Hz), 3.54 (2H, m, NHCH₂), 6.90 (1H, d, ArH, J = 9.2 Hz), 8.03 (1H, d, ArH, J = 9.2 Hz), 8.17 (1H, d, ArH, J = 9.2 Hz), 8.44 (1H, d, ArH, J = 9.2 Hz), 8.99 (1H, s, ArH), 9.03 (1H, m, NH), 9.30 (1H, s, ArH), 12.76 (1H, s, NH) | 361.0 (M + H) | 3.56 min. |
| (III) | 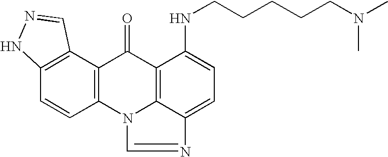 | 1.32 (2H, m, CH₂), 1.56 (2H, m, CH₂), 1.64 (2H, m, CH₂), 2.66 (6H, s, N(CH₃)₂), 2.74 (2H, m, CH₂), 2.93 (2H, m, CH₂), 6.86 (1H, d, ArH, J = 8.8 Hz), 8.01 (1H, d, ArH, J = 8.8 Hz), 8.16 (1H, d, ArH, J = 8.8 Hz), 8.45 (1H, d, ArH, J = 8.8 Hz), 8.99 (1H, s, ArH), 9.02 (1H, m, NH), 9.30 (1H, s, ArH) | 389.0 (M + H) | 4.67 min. |
| (VI) | 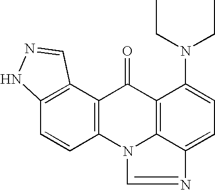 | 1.10 (6H, t, CH₃, J = 7.2 Hz), 3.52 (4H, q, CH₂, J = 7.2 Hz), 7.18 (1H, d, ArH, J = 8.8 Hz), 8.03 (1H, d, ArH, J = 8.8 Hz), 8.11 (1H, d, ArH, J = 8.8 Hz), 8.40 (1H, d, ArH, J = 8.8 Hz), 8.99 (1H, s, ArH), 9.37 (1H, s, ArH), 13.62 (1H, s, NH) | 332.2 (M + H) | 4.43 min. |
| (VII) | 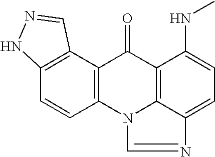 | 3.01 (3H, s, CH₃), 6.68 (1H, d, ArH, J = 8.8 Hz), 7.92 (1H, d, ArH, J = 8.8 Hz), 8.07 (1H, d, ArH, J = 8.8), 8.32 (1H, d, ArH, J = 8.8 Hz), 8.80 (1H, s, NH), 8.92 (1H, s, ArH), 9.17 (1H, s, ArH) | 290.2 (M + H) | 6.31 min. |
| (VIII) | 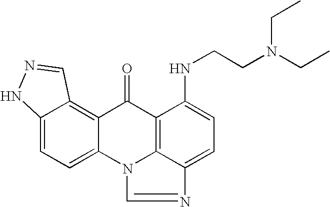 | 1.01 (6H, t, CH₃, J = 7.6 Hz), 2.56 (4H, q, CH₂, J = 7.6 Hz), 2.57 (2H, t, CH₂, J = 6.2 Hz), 3.43 (2H, m, CH₂), 6.78 (1H, d, ArH, J = 9.0 Hz), 7.94 (1H, d, ArH, J = 9.0 Hz), 8.11 (1H, d, ArH, J = 9.0 Hz), 8.36 (1H, d, ArH, J = 9.0 Hz), 8.96 (1H, s, ArH), 9.00 (1H, t, NH, J = 5.8 Hz), 9.18 (1H, s, ArH), 13.75 (1H, s, NH) | 375.4 (M + H) | 3.72 min. |
| (IX) | 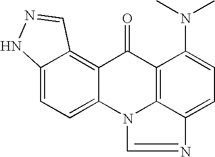 | 3.07 (6H, s, N(CH₃)₂), 7.11 (1H, d, ArH, J = 8.8 Hz), 8.01 (1H, d, ArH, J = 8.8 Hz), 8.12 (1H, d, ArH, J = 8.8 Hz), 8.42 (1H, d, ArH, J = 8.8 Hz), 8.59 (1H, s, CH), 9.37 (1H, s, CH), 13.83 (1H, s, NH) | 304.3 (M + H) | 5.70 min. |
| (X) | 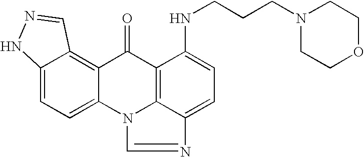 | 1.86 (2H, m, CH₂), 2.38 (4H, m, CH₂NCH₂), 2.43 (2H, t, NCH₂, J = 6.4 Hz), 3.45 (2H, m, NHCH₂), 3.62 (4H, t, CH₂OCH₂, J = 3.4 Hz), 6.87 (1H, d, ArH, J = 8.8 Hz), 7.98 (1H, d, ArH, J = 8.8 Hz), 8.12 (1H, d, ArH, J = 8.8 Hz), 8.41 (1H, d, ArH, J = 8.8 Hz), 8.99 (1H, s, CH), 9.11 (1H, m, NH), 9.25 (1H, s, ArH) | 403.3 (M + H) | 3.75 min. |
| (XI) | 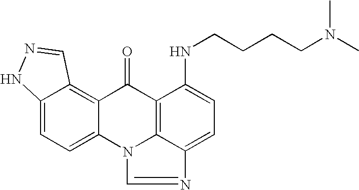 | 1.53 (2H, m, CH₂), 1.62 (2H, m, CH₂), 2.09 (6H, s, N(CH₃)₂), 2.22 (2H, m, CH₂), 3.32 (2H, m, CH₂), 6.67 (1H, d, ArH, J = 9.0 Hz), 7.86 (1H, d, ArH, J = 9.0 Hz), 8.07 (1H, d, ArH, J = 9.0 Hz), 8.33 (1H, d, ArH, J = 9.0 Hz), 8.86 (1H, t, NH, J = 5.5 Hz), 8.91 (1H, s, ArH), 9.17 (1H, s, ArH), 13.70 (1H, s, NH) | 375.2 (M + H) | 4.07 min. |
| (XII) | 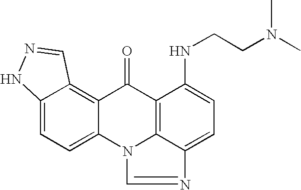 | 2.25 (6H, s, N(CH₃)₂), 2.59 (2H, t, CH₂, J = 6.2 Hz), 3.42 (2H, m, CH₂), 6.76 (1H, d, ArH, J = 9.0 Hz), 7.93 (1H, d, ArH, J = 9.0 Hz), 8.10 (1H, d, ArH, J = 9.0 Hz), 8.37 (1H, d, ArH, J = 9.0 Hz), 8.96 (1H, s, ArH), 8.98 (1H, t, NH, J = 5.8 Hz), 9.22 (1H, s, ArH), 13.80 (1H, s, NH) | 347.1 (M + H) | 3.03 min. |
| (XIV) | 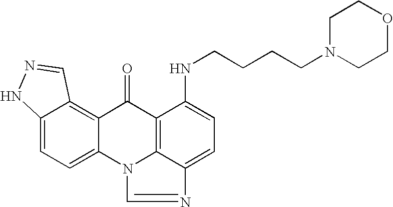 | 1.50 (4H, m, CH₂), 2.26 (2H, m, CH₂), 2.32 (4H, m, CH₂), 3.07 (2H, m, NCH₂), 3.56 (4H, m, CH₂NCH₂), 6.08 (1H, d, ArH, J = 9.8 Hz) 7.46 (1H, d, ArH, J = 9.0 Hz), 7.74 (1H, ArH, J = 9.0 Hz), 7.93 (1H, d, ArH, J = 9.0 Hz), 8.51 (1H, s, ArH), 11.66 (1H, m, NH), 12.24 (1H, s, ArH), 13.42 (1H, s, NH) | 437.2 (M + H) | 4.10 min. |
| (XV) | 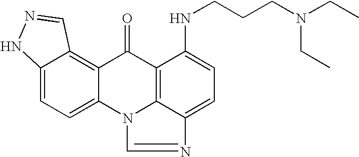 | 1.21 (6H, t, CH₃, J = 7.2 Hz), 2.12 (2H, m, CH₂), 3.12 (4H, m, CH₂NCH₂), 3.15 (2H, m, CH₂), 3.55, (2H, m, NHCH₂), 6.97 (1H d, ArH, J = 8.8 Hz), 8.01 (1H, d, ArH, J = 8.8 Hz), 8.15 (1H, d, ArH, J = 8.8 Hz), 8.41 (1H, d, ArH, J = 8.8 Hz), 8.94 (1H, s, CH), 9.59 (1H, s, CH), 10.53 (1H, s, NH) | 389.1 (M + H) | 4.12 min. |
| (XVI) | 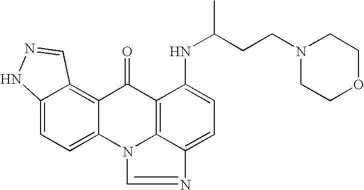 | 1.32 (3H, d, CH₃, J = 6.8 Hz), 1.77 (2H, m, CH₂), 2.38 (4H, m, CH₂NCH₂), 2.39 (2H, m, CH₂), 3.57 (4H, t, CH₂OCH₂, J = 3.4 Hz), 3.97 (1H, m, CH), 6.90 (1H, d, ArH, J = 8.8 Hz), 7.98 (1H, d, ArH, J = 8.8 Hz), 8.14 (1H, d, ArH, J = 8.8 Hz), 8.42 (1H, d, ArH, J = 8.8 Hz), 9.00 (1H, s, CH), 9.04 (1H, d, NH, J = 8.4 Hz), 9.26 (1H, s, CH), 13.69 (1H, s, NH) | 417.2 (M + H) | 4.26 min. |
| (XVII) | 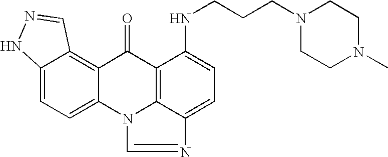 | 1.84 (2H, m, CH₂NCH₂), 2.19 (3H, s, NCH₃), 2.43-2.49 (6H, m, NCH₂), 3.44 (2H, m, NHCH₂), 6.84 (1H, d, ArH, J = 8.8 Hz), 7.97 (1H, d, ArH, J = 8.8 Hz), 8.13 (1H, d, ArH, J = 8.8 Hz), 8.41 (1H, d, ArH, J = 8.8 Hz), 9.00 (1H, s, CH), 9.04 (1H, m, NH), 9.25 (1H, s, CH), 13.72 (1H, s, NH) | 416.1 (M + H) | 4.00 min. |

### Example 2

### Determination of FLT3 and Related Protein Tyrosine Kinase Inhibitory Targeting In Vitro

Representative compounds of formula (I) or formula (Ia) are screened for activity against FLT3 and related protein tyrosine kinase in standard pharmacological test procedures. Based on the activity shown in the standard pharmacological test procedures, the compounds of the present teachings can be useful as FLT-3 inhibitors.

The terms “IC50” and “EC50” are used interchangeably. As used herein, “EC50” refers to nMolar concentration at median percent inhibition determined by dose respose (DR) assay. As used herein, the term “E1000” refers to percent inhibition at 1000 nMolar determined by assay. EC50 was calculated based on dose response curve was fitted to 4 parameter Hill equation.

Testing of the compounds described in this invention was carried out using the SelectScreen™ platform from Invitrogen, Inc. (Carlsbad. Calif., USA) and the details of its performance are readily viewed via the web by linking to: http://www.invitrogen.com/downloads/SelectScrn_Brochure.pdf.

Briefly, the approach is based on treating each specific kinase with a unique substrate and optical reporter system in the presence of ATP at 100 micromolar. In controls, the substrate is phosphorylated and a baseline optimal response is recorded.

Compounds were initially tested at 1000 nanomolar concentrations and the % inhibition of enzyme activity determined (E1000). The compounds were then re-tested by adding graded amounts of putative inhibitor which were added in 5 separate increments to generate a dose response curve. The latter is obtained by fitting to a 4 parameter Hill equation, a sigmoid saturation equation. The concentration which causes 50% enzyme inhibition (EC50) was then calculated from the dose response equation.

An effective level of inhibition in the low nanomolar range is considered to qualify the test compound as potential drug or targeting agent against the specific kinase that it has inhibited. The EC₅₀ value is, therefore, a measure of potency. Another important feature is specificity. It is considered a desirable property when claiming efficacy to determine how many kinases are inhibited by the same molecule. The fewer number inhibited points toward specificity; the greater to inhibitory promiscuity.

For the compounds of this invention, the experimental conditions were as follows. The 2× FLT3/Tyr 02 peptide mixture was prepared in 50 mM HEPES pH 7.5, 0.01 BRIJ-35, 10 mM MgCl2, 1 mM EGTA. The final 10 uL kinase reaction consists of 0.6-76.0 ng FLT3 and 2 uM Tyr 02 peptide in 50 mM HEPES pH 7.5, 0.01% BRIJ-35, 10 mM MgCl2, 1 mM EGTA. After 1 hour kinase reaction incubation, 5 uL of a 1:64 dilution of development reagent A was added.

When tested in in vitro dose response assays against FLT3, the representative compounds identified in Table I exhibited the EC50 and E1000 assay values shown in Table 2. The data was derived in the SelectScreen assay and demonstrates that low nanomolar inhibitory potency is readily achieved with this new class of compounds, in comparison to five known positive controls taken from the current pharmacopoeia of compounds recognized as active inhibitors of Flt3. Notably, the compounds of the present invention have at least comparable if not 10 fold greater potency than Symadex; the properties of Symadex as a Flt3 inhibitor and immune system modulator have been described previously by Ajami, A. M., Boss, M. A. and Paterson, J. Compounds for treating autoimmune and demyelinating diseases. US Patent Appl. 2006/0189546A1. As used herein, “Symadex” and “C-1311” are both names for the compound 5-(2-(diethylamino)ethylamino)-8-hydroxy-6H-imidazo[4,5,1-de]acridin-6-one.

We have discovered that the side chain decoration of the planar core scaffold is a key feature in determining inhibitory activity.

TABLE 2
Comparative potency of indazole class compounds of Formula I and
positive controls

FLT3
Inhibition

| | EC50 | E1000 |
|---|---|---|
| Indazole Compounds | | |
| (XVIII) | 1332 | 47 |
| (V) | 30 | 95 |
| (IV) | 14 | 98 |
| (III) | 4 | 100 |
| (VI) | 5 | 100 |
| (VII) | 5 | 99 |
| (VIII) | 69 | 87 |
| (IX) | 9 | 98 |
| (X) | 11 | 98 |
| (XI) | 2 | 100 |
| (XII) | 11 | 98 |
| (XVI) | 14 | 99 |
| (XV) | 11 | 99 |
| (XVI) | 9 | 98 |
| (XVII) | 8 | 99 |
| Positive Controls | | |
| AGL-2043 | 38 | 93 |
| GTP-14564 | 26 | 95 |
| PKC412 | 7 | 99 |
| Sunitinib | 2 | 99 |
| Symadex (C-1311) | 24 | 96 |

### Example 3

### Determination of FLT3 and Related Protein Tyrosine Kinase Specificity In Vitro

The same assay system described in Example 2 can be applied in the determination of kinase specificity. It is well understood by medicinal chemists in the development of kinase inhibitors that the resulting molecules often lack specificity. Not only are homologous kinases inhibited by the same class of compounds, but also non-homologous kinases can be affected by allosteric, induced, activated vs inactivated conformation, and protein folding interactions with the putative inhibitor, which otherwise had been designed to be specific. These off target effects are difficult to predict and can only be ruled in or out by empirically surveying a large set of kinases. It is also understood that changes to scaffolds and variation in their chemical decorations may influence the extent of promiscuity in the patterns of kinase inhibition across kinase classes.

For the compounds of this invention, a high degree of specificity as modulators of Flt3 mediated biological processes would be desirable. It would also be desirable to insure that the replacement of the phenol in Symadex with the indazole moiety should invest the new genus of molecules with similar specificity as that of their antecedent imidazoacridinine.

The following experiment was conducted to address this issue. The indazole compound (IV), described in Example 2 and representative of the new genus was tested along with Symadex against a panel of 230 kinases representative of all known kinases classes. Included in the survey were the Flt3, its mutant, and close relatives in the Class III receptor tyrosine kinase class, together with serine-threonine kinases and atypical kinases many of which share no homology of active site with Flt3 as shown, for example, in the respective “genecard” for each kinase and available for inspection at on the web at http://www.genecards.org/.

Table 3 lists the estimated EC50 values from an abridged dose response curve for Symadex (C-1311) and CF-123. The kinases are listed alphabetically by Group and Family. The results show that both the molecule representative of a new molecular class embodying the indazole pharmacophore show the same pattern of kinase inhibitory specificity as the molecule with the phenolic pharmacophore. Remarkably, the compounds of the present invention are highly specific for FLT3, and to a lesser extent V561D mutant PDGFRA (60-80 nM EC 50) and CSF-1R (200-400 nM EC50) within the broader class of tyrosine kinases identified as the “TK Group” in Table 3. Off target, and therefore unexpected activities, are also similar for both compounds, notably the inhibitory effect on the MYLK2, light chain myosin kinase, a calcium dependent, serine-threonine kinase with no active site relationship to the Flt3 kinase class.

TABLE 3
Kinase inhibitory activity (EC50 nM) survey for specificity:
Symadex (C-1311) compared to the indazole compound (IV).

| | | Symadex | |
|---|---|---|---|
| Kinase | Group | (C-1311) | (IV) |
| AKT1 (PKB alpha) | AGC | >5k | >5k |
| AKT2 (PKB beta) | AGC | >5k | >5k |
| AKT3 (PKB gamma) | AGC | >5k | >5k |
| CDC42 BPA (MRCKA) | AGC | >5k | >5k |
| CDC42 BPB (MRCKB) | AGC | >5k | 3632 |
| ROCK1 | AGC | >5k | 487 |
| ROCK2 | AGC | >5k | 963 |
| ADRBK1 (BARK1, GRK2) | AGC | >5k | >5k |
| ADRBK2 (BARK2, GRK3) | AGC | >5k | >5k |
| GRK4 | AGC | >5k | >5k |
| GRK5 | AGC | >5k | >5k |
| GRK6 | AGC | >5k | >5k |
| GRK7 | AGC | >5k | >5k |
| PRKX | AGC | 3560 | 1399 |
| PDK1 | AGC | >5k | 2898 |
| PRKACA (PKA, PKACA) | AGC | >5k | >5k |
| PRKCA (PKC alpha) | AGC | >5k | >5k |
| PRKCB1 (PKC beta I) | AGC | >5k | >5k |
| PRKCB2 (PKC beta II) | AGC | >5k | >5k |
| PRKCD (PKC delta) | AGC | >5k | >5k |
| PRKCE (PKC epsilon) | AGC | >5k | 2517 |
| PRKCG (PKC gamma) | AGC | 1895 | >5k |
| PRKCH (PKC eta) | AGC | >5k | 2126 |
| PRKCI (PKC iota) | AGC | >5k | >5k |
| PRKCQ (PKC theta) | AGC | >5k | >5k |
| PRKCZ (PKC zeta) | AGC | >5k | >5k |
| PRKG1 (PKG1) | AGC | >5k | >5k |
| PRKG2 (PKG2) | AGC | 2851 | 1908 |
| PKN1 (PRK1) | AGC | 4754 | 1388 |
| RPS6KA1 (RSK1) | AGC | 2071 | 1735 |
| RPS6KA2 (RSK3) | AGC | 460 | 840 |
| RPS6KA3 (RSK2) | AGC | 692 | 1489 |
| RPS6KA4 (MSK2) | AGC | >5k | >5k |
| RPS6KA5 (MSK1) | AGC | >5k | >5k |
| RPS6KA6 (RSK4) | AGC | 182 | 655 |
| RPS6KB1 (p70S6K) | AGC | >5k | 2370 |
| SGK (SGK1) | AGC | 2712 | 3453 |
| SGK2 | AGC | >5k | >5k |
| SGKL (SGK3) | AGC | >5k | >5k |
| EEF2K | Atypical | >5k | >5k |
| FRAP1 (mTOR) | Atypical | >5k | >5k |
| CAMK1D (CaMKI delta) | CAMK | 3996 | 2782 |
| CAMK4 (CaMKIV) | CAMK | >5k | >5k |
| CAMK2A (CaMKII alpha) | CAMK | >5k | 789 |
| CAMK2B (CaMKII beta) | CAMK | >5k | >5k |
| CAMK2D (CaMKII delta) | CAMK | 2063 | 218 |
| AMPK A1/B1/G1 | CAMK | 3478 | >5k |
| AMPK A2/B1/G1 | CAMK | 1787 | 1572 |
| BRSK1 (SAD1) | CAMK | 1934 | 1395 |
| CHEK1 (CHK1) | CAMK | >5k | >5k |
| MARK1 (MARK) | CAMK | 3191 | >5k |
| MARK2 | CAMK | 2023 | 3488 |
| PASK | CAMK | 4320 | 1226 |
| DAPK3 (ZIPK) | CAMK | >5k | 973 |
| DCAMKL2 (DCK2) | CAMK | >5k | >5k |
| MAPKAPK2 | CAMK | >5k | >5k |
| MAPKAPK3 | CAMK | >5k | >5k |
| MAPKAPK5 (PRAK) | CAMK | >5k | >5k |
| MYLK2 (skMLCK) | CAMK | 41 | 15 |
| PHKG1 | CAMK | 2204 | 1011 |
| PHKG2 | CAMK | >5k | 3716 |
| PIM1 | CAMK | 959 | 3422 |
| PIM2 | CAMK | >5k | >5k |
| PRKCN (PKD3, PRKD3) | CAMK | 221 | 302 |
| PRKD1 (PKC mu, PKD1) | CAMK | 217 | 494 |
| PRKD2 (PKD2) | CAMK | 292 | 759 |
| CHEK2 (CHK2) | CAMK | >5k | >5k |
| STK22B (TSSK2) | CAMK | >5k | >5k |
| STK22D (TSSK1) | CAMK | 1948 | 2016 |
| CSNK1A1 (CK1 alpha 1) | CK1 | 3479 | >5k |
| CSNK1D (CK1 delta) | CK1 | 673 | 4805 |
| CSNK1E (CK1 epsilon) | CK1 | 1089 | >5k |
| CSNK1G1 (CK1 gamma 1) | CK1 | >5k | >5k |
| CSNK1G2 (CK1 gamma 2) | CK1 | >5k | >5k |
| CSNK1G3 (CK1 gamma 3) | CK1 | >5k | >5k |
| CDK1/cyclin B (CDC2) | CMGC | >5k | 3947 |
| CDK2/cyclin A | CMGC | >5k | 822 |
| CDK5/p25 | CMGC | >5k | 1582 |
| CDK5/p35 | CMGC | >5k | 2802 |
| CLK1 | CMGC | 2178 | 444 |
| CLK2 | CMGC | 315 | 55 |
| CLK3 | CMGC | >5k | >5k |
| DYRK1A | CMGC | 708 | 468 |
| DYRK1B | CMGC | 991 | 820 |
| DYRK3 | CMGC | 611 | 380 |
| DYRK4 | CMGC | >5k | >5k |
| HIPK1 (Myak) | CMGC | >5k | 1215 |
| HIPK4 | CMGC | 1825 | 1310 |
| GSK3A (GSK3 alpha) | CMGC | >5k | 3620 |
| GSK3B (GSK3 beta) | CMGC | >5k | >5k |
| MAPK1 (ERK2) | CMGC | >5k | >5k |
| MAPK10 (JNK3) | CMGC | >5k | 2810 |
| MAPK11 (p38 beta) | CMGC | >5k | >5k |
| MAPK12 (p38 gamma) | CMGC | >5k | >5k |
| MAPK13 (p38 delta) | CMGC | >5k | >5k |
| MAPK14 (p38 alpha) | CMGC | 2967 | 3787 |
| MAPK3 (ERK1) | CMGC | >5k | >5k |
| MAPK8 (JNK1) | CMGC | >5k | >5k |
| MAPK9 (JNK2) | CMGC | >5k | >5k |
| SRPK1 | CMGC | >5k | >5k |
| SRPK2 | CMGC | >5k | >5k |
| STK23 (MSSK1) | CMGC | >5k | >5k |
| AURKA (Aurora A) | Other | >5k | >5k |
| AURKB (Aurora B) | Other | >5k | >5k |
| AURKC (Aurora C) | Other | >5k | >5k |
| CSNK2A1 (CK2 alpha 1) | Other | >5k | >5k |
| CSNK2A2 (CK2 alpha 2) | Other | 4803 | >5k |
| IKBKB (IKK beta) | Other | >5k | 2803 |
| TBK1 | Other | >5k | >5k |
| NEK1 | Other | 3381 | >5k |
| NEK2 | Other | >5k | >5k |
| NEK4 | Other | >5k | >5k |
| NEK6 | Other | >5k | >5k |
| NEK9 | Other | >5k | 2927 |
| PLK1 | Other | >5k | >5k |
| PLK2 | Other | >5k | >5k |
| PLK3 | Other | >5k | >5k |
| MAP3K8 (COT) | STE | 4628 | >5k |
| MAP4K2 (GCK) | STE | 4926 | >5k |
| MAP4K4 (HGK, ZC1) | STE | 3132 | >5k |
| MAP4K5 (KHS1) | STE | 2429 | >5k |
| MINK1 (ZC3) | STE | 1088 | 1364 |
| MST4 | STE | 1580 | >5k |
| PAK2 (PAK65) | STE | >5k | >5k |
| PAK3 | STE | >5k | >5k |
| PAK4 | STE | >5k | >5k |
| PAK6 | STE | >5k | >5k |
| PAK7 (KIAA1264, PAK5) | STE | >5k | >5k |
| STK24 (MST3) | STE | 733 | 1041 |
| STK25 (YSK1) | STE | >5k | >5k |
| STK3 (MST2) | STE | >5k | >5k |
| STK4 (MST1) | STE | >5k | 2166 |
| TAOK2 (TAO1) | STE | >5k | >5k |
| MAP2K1 (MEK1) | STE | >5k | >5k |
| MAP2K2 (MEK2) | STE | >5k | >5k |
| MAP2K6 (MKK6) | STE | >5k | >5k |
| ABL1 | TK | >5k | 3777 |
| ABL1 E255K | TK | >5k | 3720 |
| ABL1 G250E | TK | >5k | 4050 |
| ABL1 T315I | TK | >5k | >5k |
| ABL1 Y253F | TK | 4485 | 2524 |
| ABL2 (Arg) | TK | 3207 | 2874 |
| ALK | TK | 2475 | 4629 |
| LTK (TYK1) | TK | >5k | >5k |
| AXL | TK | 3772 | 4442 |
| MERTK (cMER) | TK | 3601 | >5k |
| TYRO3 (RSE) | TK | 2438 | 3573 |
| CSK | TK | >5k | >5k |
| MATK (HYL, CTK) | TK | >5k | >5k |
| EGFR (ErbB1) | TK | >5k | >5k |
| EGFR (ErbB1) L858R | TK | >5k | >5k |
| EGFR (ErbB1) L861Q | TK | >5k | >5k |
| EGFR (ErbB1) T790M | TK | 2899 | 4007 |
| ERBB2 (HER2) | TK | >5k | >5k |
| ERBB4 (HER4) | TK | >5k | >5k |
| EPHA1 | TK | >5k | >5k |
| EPHA2 | TK | >5k | >5k |
| EPHA3 | TK | >5k | >5k |
| EPHA4 | TK | >5k | >5k |
| EPHA5 | TK | >5k | >5k |
| EPHA8 | TK | 3346 | 4151 |
| EPHB1 | TK | >5k | >5k |
| EPHB2 | TK | >5k | >5k |
| EPHB3 | TK | >5k | >5k |
| EPHB4 | TK | >5k | >5k |
| PTK2B (FAK2, PYK2) | TK | >5k | >5k |
| FER | TK | >5k | >5k |
| FES (FPS) | TK | 1050 | 2254 |
| FGFR1 | TK | 2388 | 3575 |
| FGFR2 | TK | 4498 | 2855 |
| FGFR3 | TK | >5k | 3211 |
| FGFR3 K650E | TK | >5k | >5k |
| FGFR4 | TK | >5k | >5k |
| IGF1R | TK | >5k | 1561 |
| INSR | TK | >5k | 2396 |
| INSRR (IRR) | TK | >5k | 1149 |
| JAK1 | TK | >5k | >5k |
| JAK2 | TK | >5k | >5k |
| JAK2 JH1 JH2 | TK | >5k | >5k |
| JAK2 JH1 JH2 V617F | TK | >5k | >5k |
| JAK3 | TK | >5k | >5k |
| MET (cMet) | TK | >5k | >5k |
| MET M1250T | TK | >5k | >5k |
| MST1R (RON) | TK | >5k | >5k |
| MUSK | TK | 3713 | 1432 |
| CSF1R (FMS) | TK | 428 | 205 |
| FLT3 | TK | 15 | 8 |
| FLT3 D835Y | TK | 3 | 1 |
| KIT | TK | 2250 | 1100 |
| KIT T670I | TK | >5k | >5k |
| PDGFRA (PDGFR alpha) | TK | 1234 | 562 |
| PDGFRA D842V | TK | 1057 | 370 |
| PDGFRA T674I | TK | >5k | >5k |
| PDGFRA V561D | TK | 83 | 60 |
| PDGFRB (PDGFR beta) | TK | 1793 | 278 |
| RET | TK | 1123 | 587 |
| RET V804L | TK | 735 | 864 |
| RET Y791F | TK | 1237 | 627 |
| ROS1 (ROS) | TK | >5k | 2657 |
| BLK | TK | 220 | 481 |
| FGR | TK | 1268 | 367 |
| FRK (PTK5) | TK | 2110 | 389 |
| FYN | TK | >5k | 754 |
| HCK | TK | 3983 | 2282 |
| LCK | TK | 824 | 345 |
| LYN A | TK | 791 | 389 |
| LYN B | TK | 812 | 426 |
| PTK6 (Brk) | TK | >5k | >5k |
| SRC | TK | 4411 | 437 |
| SRC N1 | TK | 3154 | 511 |
| SRMS (Srm) | TK | >5k | >5k |
| YES1 (YES) | TK | >5k | 1575 |
| SYK | TK | 3910 | >5k |
| ZAP70 | TK | >5k | >5k |
| BMX | TK | >5k | 3225 |
| BTK | TK | 3021 | 1440 |
| ITK | TK | >5k | 637 |
| TEK (Tie2) | TK | >5k | >5k |
| NTRK1 (TRKA) | TK | 1434 | 438 |
| NTRK2 (TRKB) | TK | >5k | 780 |
| NTRK3 (TRKC) | TK | 3725 | 240 |
| FLT1 (VEGFR1) | TK | >5k | >5k |
| FLT4 (VEGFR3) | TK | 3962 | 1495 |
| KDR (VEGFR2) | TK | 1601 | 830 |
| IRAK4 | TKL | 847 | 3321 |
| MAP3K9 (MLK1) | TKL | >5k | >5k |
| BRAF | TKL | 4451 | 1443 |
| BRAF V599E | TKL | 3404 | 1167 |
| RAF1 (cRAF) Y340D Y341D | TKL | >5k | >5k |
| ACVR1B (ALK4) | TKL | >5k | >5k |

### Example 4

### Determination of Growth Inhibitory Activity Against FLT3 Expressing Cells by Cellular Proliferation Assay

The viability of Flt3 expressing cells can be evaluated using a tetrazolium salt reduction cell-based assay. In viable cells, notably among them various immortalized lymphoid and myelioid lines, this colorimetric assay can measure mitochondrial reduction of a tetrazolium component (MTS) into an insoluble formazan product. BaF3, both wild type and mutant, as well as transfected strains, MV4-11 and MOLM14 are well-characterized Flt3-dependent humanized or human cell lines which express active Flt3 receptors (see, Yee et al. Blood (2002)100 (8):2941-2949; Levis et al. (2002) 99(11): 3885-3891). These cell lines were used to determine the ability of the compounds provided herein to inhibit Flt3 in intact cells. In each case, and by methods known to those practiced in the art of cell culture, proliferation was measured after 72 hour incubation with the compounds provided herein using a standard MTS protocol (Promega Cat #5430 “Cell Titer 96 Aqueous Non-radioactive Cell Proliferation Assay”).

In general, cells were plated at 10,000-20,000 cells per well in DMEM medium with 0.5% serum. The compound plate was set up by aliquoting into column 1 of a 96 well 300 ul polypropylene plate, the negative control (DMSO), aliquoting into column 12 the positive control and titrating the test compound in serial dilutions into columns 2-1 l. An aliquot from each well of the compound plate was transferred to the plated cells and then incubated @ 370 C in 5% CO2. [00538] MTS tetrazolium compound (Owen's reagent) was thawed in a H2O bath. 20 μl of MTS tetrazolium was added to each well of optical plate and the cells were incubated @ 37° C. in 5% CO2 for 2 hours. The absorbance measured at 490 nm using a microplate reader. Cell proliferation values are measured in terms of concentration of test compound that achieves 50% inhibition of cellular proliferation compared to control (IC50) and are reported in Table 4.

These results show that the compounds of this invention inhibit the growth of Flt3 expressing cells in vivo. Once again note is drawn to the efficacy of (IV), a representative indazole class molecule, and its favorable comparison to the phenolic substituted imidazoacridinone, Symadex.

TABLE 4
Growth inhibitory activity (in vivo EC50 nM) on Flt3 expressing
cells

| | BaF3- | | | | | |
|---|---|---|---|---|---|---|
| | FLT3- | BaF3- | BaF3- | BaF3- | MV- | |
| DRUG | WT + IL-3 | ITD | D835Y | D835N | 4-11 | MOLM14 |
| (V) | 246 | 90 | 172 | — | — | 333 |
| (IV) | 6 | 20 | 13 | 12 | 12 | — |
| (III) | 752 | 223 | 887 | 316 | 184 | — |
| (VI) | 1391 | 74 | 171 | — | — | 84 |
| (VII) | 356 | 36 | 158 | — | — | 60 |
| (VIII) | 341 | 30 | 129 | — | — | 627 |
| (IX) | >2000 | 80 | 723 | — | — | 82 |
| (X) | 492 | 35 | 79 | — | — | 90 |
| Symadex | 13.2 | 11.2 | 8.9 | 14.3 | 13.1 | — |

Examples 5 through 9, below, present further evidence of efficacy of the compounds disclosed herein. Table 5 lists the compounds used in the assays described in Examples 5 through 9 and summarizes the findings.

TABLE 5

| | | | | | | IC₅₀ SRB assay | | | | |
|---|---|---|---|---|---|---|---|---|---|---|
| | | | | | | Mda | | | Mi- | |
| | | EC₅₀^{(a)} | | | | MB- | MV4- | Raw | gra- | |
| | | | Flt3- | CSF- | | 231 | 11 | 264.7 | tion | BrdU^{(b)} |
| No | Structure | Flt3 | D835Y | 1R | MLCK | μm | nm | nm | μm | μm |
| SD^{(c)} | 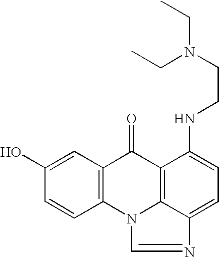 | 40 | 24 | 430 | 95 | 6.8 | 12 | ND | 10 | ND |
| (IV) | 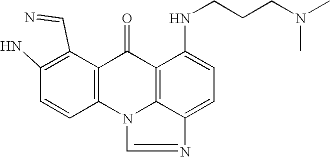 | 14 | 12 | 200 | 21 | 5.4 | <1 | <0.1 | 1 | 2.5 |
| (X) | 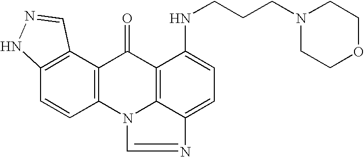 | 11 | 11 | 280 | 52 | ND | 2 | 2.7 | 5 | 20 |
| (XI) | 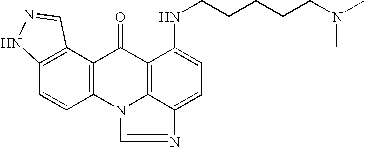 | 5 | 7 | 153 | 18 | 5.2 | <1 | ND | 2.5 | 10 |
| (XII) | 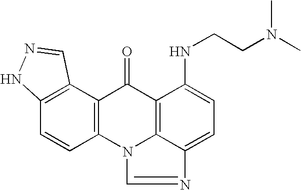 | 5 | 3 | 360 | 18 | 7.4 | <1 | <0.1 | >5 | 2.5 |
| (XIV) | 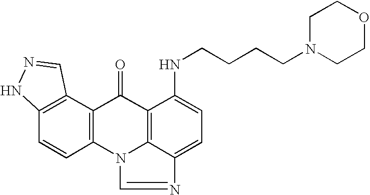 | 14 | 7 | 971 | 86 | ND | 35 | 2.1 | 2.5 | ND |
| (XV) | 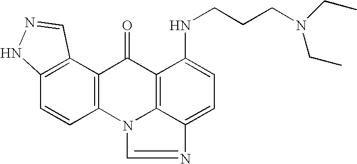 | 11 | 4 | 490 | 8 | 4 | <1 | <0.1 | 1 | ND |
| (XVII) | 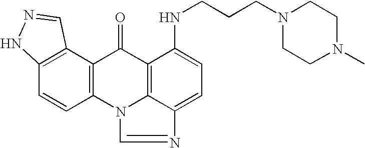 | 15 | 5 | 950 | 9 | 45.3 | 82 | 2.6 | 5 | >20 |
| (XVI) | 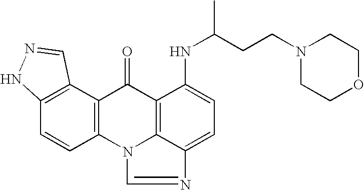 | 9 | 8 | 245 | 53 | 5.1 | 121 | 25.68 | 1 | 5 |

^{(a)}from 10 pt curve or estimate based on 1 μm inhibition assay at ATP Kmapp;
^{(b)}Inhibition of cell proliferation;
^{(c)}Symadex

### Example 5

### Inventive Compounds are Highly Cytotoxic to Cell Lines Expressing FLT3

The cytotoxic activity of compounds listed in Table 5 was determined with a sulforhodamine B (SRB) assay on panel of cell lines, including MdaMb231 (breast cancer), MV4-11 (leukemia), PC-3 (prostate cancer), U266B1 (multiple myeloma), C7 macrophages, and Raw264.7 (murine macrophage-like cells).

Cells growing in relevant media were seeded in a 96 well plate and left to adhere overnight. Cells were plated at appropriate cell densities:
MV4-11: 20.000 cells/well
Mda-Mb231: 6000 cells/well
C7: 10.000 cells/well
Raw264.7: 2000 cells/well
PC-3: 6000 cells/well
U266B1: 40.000 cells/well.

Cells were incubated before the compounds were added the next day to a top concentration of 100 μM and serially diluted 1 in 5 across the plate. After a 3 day incubation period (6 days for C7 and Raw264.7) cells were fixed with trichloroacetic acid (TCA) to end concentration of 10% for adherent cells and 16% end concentration for suspension cells for 1 hr at 4° C. before washing. Cells were stained with 0.4% sulforhodamine-B dye (SRB) in 1% acetic acid for 30 minutes and washed in 1% acetic acid. To solubilize the dye 100 μl 10 mM Tris, pH 10.5 was added and the absorbance was read at 564 nm. IC₅₀ values were generated using the SoftMAX Pro™ software. The data is presented below in Tables 6 through 11.

TABLE 6
IC₅₀ of Compounds of Table 5 Measured Against MV4-11 Cell Line

IC₅₀ values

100 μM highest concentration, diluted 1:5

| | |
|---|---|
| Symadex | 12 nM |
| (IV) | <1 nM |
| (X) | 2 nM |
| (XI) | <1 nM |
| (XII) | <1 nM |
| (XVI) | 121 nM |

50 μM highest concentration, diluted 1:4

| | |
|---|---|
| Symadex | 28 nM |
| (XIV) | 35 nM |
| (XV) | <1 nM |
| (XVII) | 82 nM |

TABLE 7
IC₅₀ of Compounds of Table 5 Measured Against MdaMb231
Cell Line

IC₅₀

100 μM highest concentration, diluted 1:5

| | |
|---|---|
| Symadex | 6.8 μM |
| (XIV) | ND |
| (XV) | 4.0 μM |
| (XVII) | 45.3 μM |

100 μM highest concentration, diluted 1:5

| | |
|---|---|
| Symadex | 22.0 μM |
| (IV) | 5.4 μM |
| (X) | ND |
| (XI) | 5.2 μM |
| (XII) | 7.4 μM |
| (XVI) | 5.1 μM |

TABLE 8
IC₅₀ of Compounds of Table 5 Measured Against PC-3 Cell Line

IC₅₀

100 μM highest concentration, diluted 1:5

| | |
|---|---|
| Symadex | ND |
| (IV) | 2.97 μM |
| (X) | 17.3 μM |
| (XI) | 3.28 μM |
| (XII) | ND |
| (XVI) | 8.92 μM |

100 μM highest concentration, diluted 1:5

| | |
|---|---|
| Symadex | ND |
| (XIV) | 56.3 μM |
| (XV) | 32.81 μM |
| (XVII) | 9.65 μM |

TABLE 9
IC₅₀ of Compounds of Table 5 Measured Against U266B1 Cell Line

IC50

100 μM highest concentration, diluted 1:5

| | |
|---|---|
| Symadex | ND |
| (IV) | 6.94 μM |
| (X) | 7.06 μM |
| (XI) | 12.4 μM |
| (XII) | 6.11 μM |
| (XVI) | 14.53 μM |

TABLE 10
IC₅₀ of Compounds of Table 5 Measured Against C7 Cell Line (6 day
assay)

IC₅₀

5 μM highest concentration, diluted 1:4

| | |
|---|---|
| Symadex | 15 nM |
| (XIV) | 95 nM |
| (XV) | 13 nM |
| (XVII) | 360 nM |

TABLE 11
IC₅₀ of Compounds of Table 5 Measured Against Raw264.7 Cell Line

IC₅₀ values

25 μM highest concentration, diluted 1:5

| | |
|---|---|
| Symadex | ND |
| (XIV) | 2.1 nM |
| (XV) | ND |
| (XVII) | 2.6 nM |
| (IV) | ND |

25 μM highest concentration, diluted 1:5

| | |
|---|---|
| (X) | 2.7 nM |
| (XI) | ND |
| (XII) | ND |
| (XVI) | 25.68 nM |
| Sutent | 763.1 nM |

The data presented in Tables 6 through 11 demonstrates that the inventive compounds of the present invention exhibit micromolar activity against solid tumour cell lines, and nanomolar activity against cell lines expressing FLT3. Like Symadex, the representative compounds demonstrated the highest efficacy against MV4-11 cells (containing ITD mutation in the Flt3 receptor gene), C7 and Raw264.7 cells.

### Example 6

### Inventive Compounds Inhibit Metastatic Ability of Tumor Cells by Inhibiting Cell Motility

Compounds of Table 5 were tested in comparison with Symadex and Sutent™ (sunitinib) for their ability to inhibit the migration of MdaMb231 cells. The in vitro scratch assay is used to determine the ability of cancer cells to migrate into and close an open wound; such migration is considered to have a role in metastasis of tumor cells.

Cells were plated in gelatin-coated 6-well plates and grown until a confluent monolayer was formed. When confluent, a straight scratch was made with a p200 pipette tip, cells were washed and compounds were added at concentrations of 1 μM, 2.5 μM, 5 μM, 10 μM and 20 μM. Images were taken at specific reference points at 0, 24 and 48 hours with Zeiss Axiovert 40 CFL microscope and Axiocam MRc5 camera and the ability of the cells to move in and close the wound was measured.

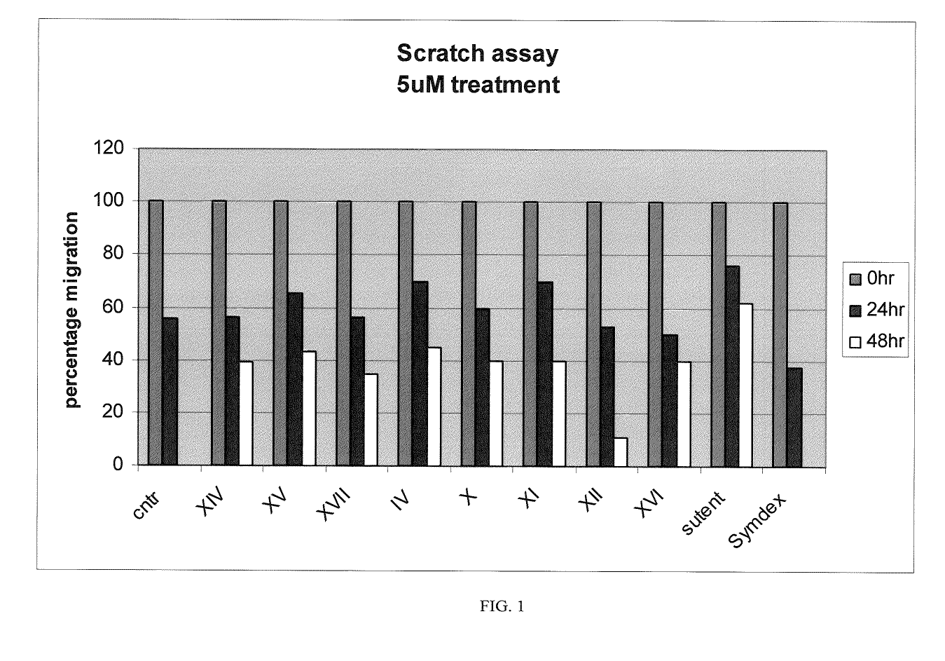

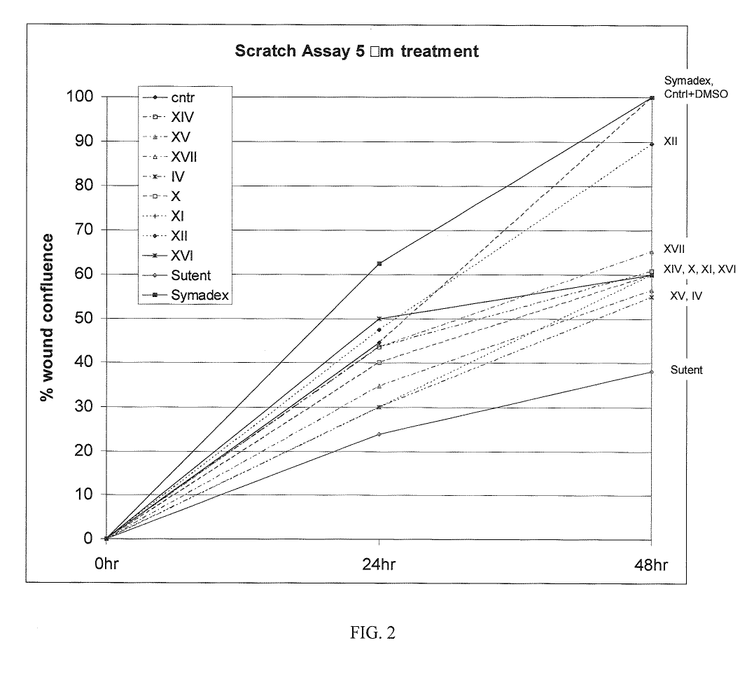

The data is presented in FIG. 1, which is a bar plot of percent cell migration into the “wound” in each sample (measured with respect to the control) and FIG. 2, which is a plot of percent wound confluence as a function of time (measured with respect to the control).

The data supports the conclusion that treatment with Compounds (XVII), (IV), (X), (XII) or (XVI) at 5 μM resulted in inhibition of cell motility after 48 hrs, while 2.5 μM of Compound (XIV) or 1 μM of Compound (XV) was sufficient to inhibit migration of Mda231 cells after 48 hours. For comparison, Symadex inhibits cell migration of Mda231 cells at 10 μM. Thus, the representative compounds of the present invention possess low IC50 in an assay that is an in vitro model for tumor metastasis.

### Example 7

### Inventive Compounds Inhibit Metastatic Ability of Tumor Cells by Inhibiting Cell Invasiveness

Representative compounds were tested for their ability to inhibit invasion of Mda231 cells. Invasive cells are a mark of metastatic tumors. The in vitro invasion assay is based on the Boyden chamber assay principle.

The 96-well BME cell invasion assay kit was used according to the manufacturers' protocol (Culturex™ catalog number: 3455-096-K). Prior to assay cells were serum starved for 18 hrs and the membrane of the top invasion chamber was coated with 50 μl of 0.83× BME solution and incubated overnight at 37° C./5% CO₂. The next day cells were harvested and 40.000 cells/well in 50 μl medium without serum were plated to top chamber. 150 μl of medium with serum was added to bottom chamber. Compounds were added and cells were incubated for 24 hrs at 37° C./5% CO₂. The remaining cells were assayed for standard curve.

After 24 hours, the bottom chamber was aspirated and washed twice with 200 μl wash buffer. The bottom chamber was transferred to a black assay plate. Cells migrated through the membrane are stained with Calcein-AM and measured with fluorescence spectrophotometry; 150 μl Calcein-AM/Cell dissociation solution was added and incubated at 37° C./5% CO₂. After 1 hour, the plate was read at 485 nm excitation, 520 nm emission. Using standard curve, the fluorescence signal measured was converted to cell number and percent invasion was determined.

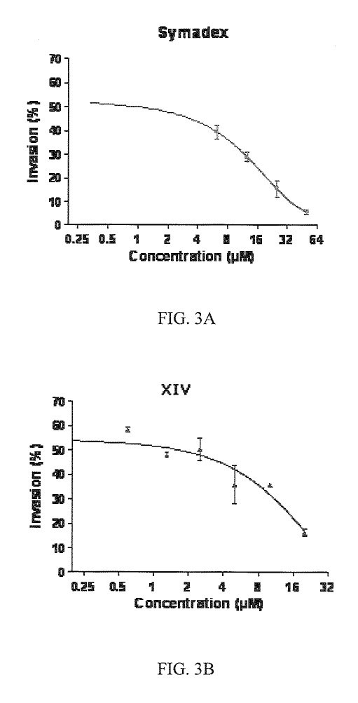

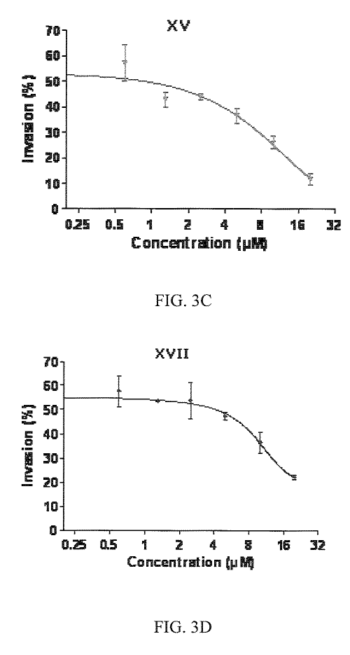

The data is presented in FIGS. 3A through 3D, which are the plots of percent invasion as a function of concentration of the indicated test compounds. A negative control exhibited 52% invasion.

The data demonstrates that the test compounds inhibit invasion of Mda231 cells after 24 hours. Compounds (XIV), (XV), and (XVII) have similar efficacy and are slightly more effective than Symadex at inhibiting cell invasion.

### Example 8

### Inventive Compounds Possess Anti-Proliferative Activity

Representative compounds of the present invention were evaluated in a BrdU assay to determine their effect on the cell proliferation of MdaMb231 cells.

The effect of the inventive compounds on cell proliferation was analyzed by detecting 5-Bromo-2′-deoxy-uridine (BrdU) incorporation after 24 hour treatment with the test compounds. The cell proliferation ELISA BrdU kit (Roche™ cat. No. 11 647 229 001) was used according to manufacturer's protocol. Cells were plated at 10.000 cells/well. The next day compounds were added in concentration range of 2.5, 5, 10 and 20 μM.

After 24 hours, 12 μl/well BrdU labelling solution was added and cells were incubated for 6 hours at 37° C./5% CO₂ before labeling solution was removed. Cells were fixed with 200 μl FixDenat™ solution for 30 minutes. After removal of fixative 100 μl BrdU-peroxidase working solution was added and cells were incubated for 90 minutes. Cells were subsequently washed three times. 100 μl substrate solution was added and after 30 minutes the absorbance was read at 370 nm.

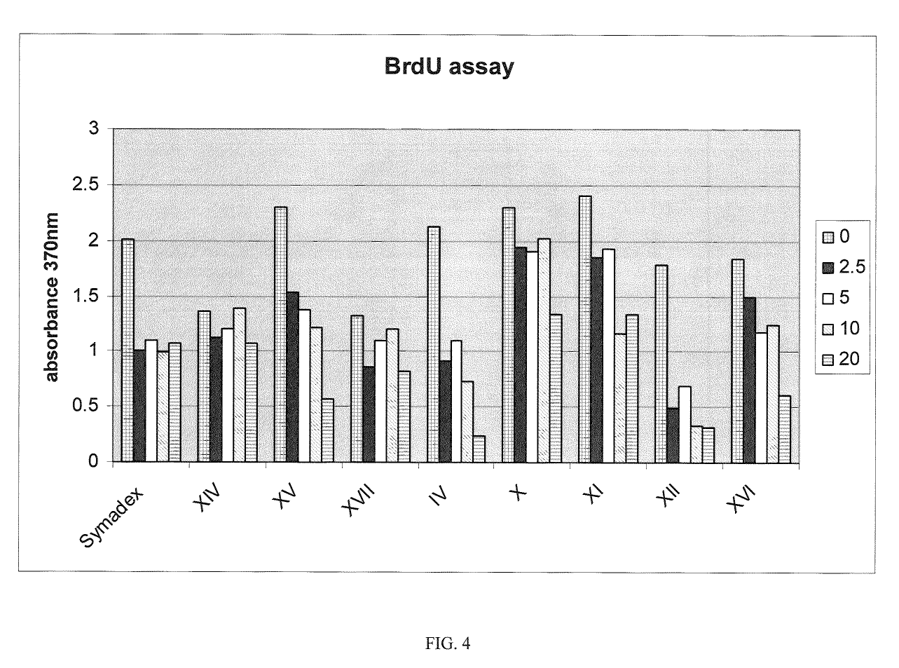

The data is presented in FIG. 4, which is a bar plot of absorbance of the cell cultures at 370 nm by each sample, each sample including the specified test compound. The data demonstrates that compounds (IV) and (XII) inhibited cell proliferation at a concentration of 2.5 μM, while compounds (XV), (X), (XI), and (XVI) inhibited cell proliferation at a concentration of 20 μM.

### Example 9

### Inventive Compounds Inhibit In Vitro Osteoclast Differentiation that Plays a Role in Bone Metastasis

Bone, as well as lung and liver, is one of the most preferential metastatic sites for common cancers, including breast, lung and prostate cancers. Bone metastases are frequently associated with severe bone pain that greatly diminish quality of life for patients. In the process of bone metastasis, metastatic tumour cells in bone enhance bone resorption (osteolysis) by inducing and activating osteoclasts. This in turn releases growth factors, such as TGF-β and IGF-1 from the bone matrix that promote tumour growth, resulting in a vicious cycle. Therefore suppression of hyperactive osteoclasts (OCL), and thus osteolysis, may be effective against bone metastasis.

Osteoclasts are multinucleated cells formed by fusion of Flt3-positive monocyte-macrophage cells, which can also differentiate to dendritic cells and macrophages. Differentiation of osteoclasts is directed by osteoblasts and stromal cells in hone microenvironment. Osteoblasts and stromal cells secrete RANKL, a member of the TNF superfamily, and M-CSF, which are essential for osteoclast differentiation and maturation in bone.

Without being bound to any particular theory, it is believed that the compounds of the invention target MLCK and M-CSF in addition to Flt3. MLCK is an important protein for cell migration, while M-CSF is essential for OCL differentiation.

Representative compounds of the invention were evaluated to determine whether differentiation of OCL is affected after treatment in an OCL differentiation assay using Raw264.7 cells. Raw264.7 cells are murine macrophage-like cells that can differentiate into multinucleated osteoclasts (OCL) when soluble RANKL and M-CSF are added to culture medium.

2500 cells/well were plated on gelatin-coated LabTek™ Chamber slides. After 24 hours, cells were incubated either with control medium or with medium including 10 ng/ml M-CSF and 50 ng/ml soluble RANKL. Compounds were added and cells were incubated for 6 days. Medium and the compounds were refreshed every 3 days.

After 6 days, tartrate resistant acid phosphatase (TRAP) staining was performed according to manufacturers' protocol (Sigma™ cat. nr. 387A-1kt). Briefly, cells were fixed for 30 seconds in citrate solution containing acetone and formaldehyde. Cells were washed before Fast Garnet™ GBC staining solution was added and incubated at 37° C. for 1 hour. Subsequently, cells were washed again. Hematoxylin counterstaining was done for 2 minutes. Slides were dried and sections were analysed with Axioskop 40 microscope. Cells were defined as osteoclasts when they contained more than three nuclei.

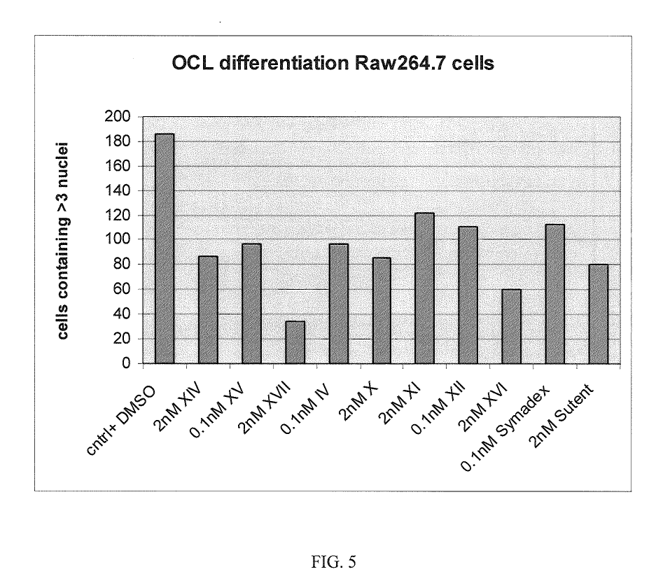

The data is presented in FIG. 5, which is a bar plot of the number of cell in each sample containing more than three nuclei. The data demonstrates that the test compounds are potent inhibitors of Raw264.7 cells and have an effect on in vitro OCL differentiation at low concentrations.

While this invention has been particularly shown and described with references to preferred embodiments thereof, it will be understood by those skilled in the art that various changes in form and details may be made therein without departing from the scope of the invention encompassed by the appended claims.

1. A compound of formula (I) or a pharmaceutically acceptable salt thereof: 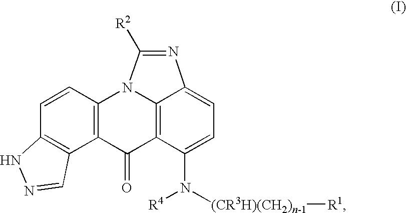
wherein:
R¹ is H or NR^{a}R^{b};
R² is H or C1-C4 alkyl;
R³ is H or C1-C4 alkyl;
R⁴ is H or C1-C4 alkyl;
n is an integer from 2 to 5; and
R^{a} and R^{b}, each independently are hydrogen or an optionally substituted alkyl; or
R^{a} and R^{b}, taken together with the nitrogen to which they are attached, form a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d};
wherein R^{c} and R^{d} are independently H, methyl or ethyl.

2. The compound of claim 1, wherein R¹ is H.

3. The compound of claim 1, wherein R¹ is NR^{a}R^{b}.

4. The compound of claim 3, wherein R^{a} and R^{b}, each independently are hydrogen or an optionally substituted alkyl.

5. The compound of claim 3, wherein R^{a} and R^{b}, taken together with the nitrogen to which they are attached, form a non-aromatic heterocycle, optionally substituted at one or more substitutable carbon atoms with methyl, hydroxyl, or methoxy, and optionally N′-substituted with C1-C4 alkyl or C1-C4 alkyl substituted with NR^{c}R^{d}.

6. The compound of claim 1, wherein the compound is represented by formula (Ia) or a pharmaceutically acceptable salt thereof:

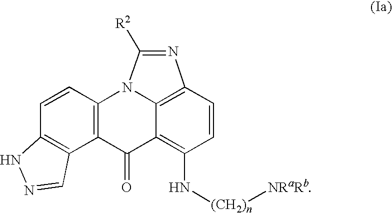

7. The compound of claim 1, wherein
R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; or
R^{a} and R^{b} or individually are hydrogen or a C1-C3 alkyl optionally substituted with —OH, —SH, halogen, cyano, nitro, amino, —COON, a C1-C3 alkyl.

8. The compound of claim 7, wherein
R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}.

9. The compound of claim 8, represented by the following formula:

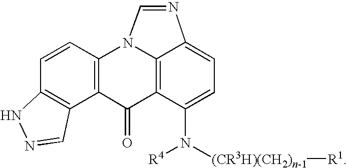

10. The compound of claim 9 wherein n is 2 or 3.

11. The compound of claim 10, wherein R^{a} and R^{b}, taken together with the nitrogen to which they are attached, form a 5-7 member non-aromatic heterocycle selected form a group consisting of 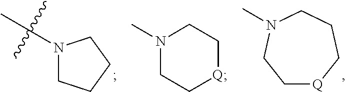
wherein Q is S, O, CH₂, NH, or NR¹⁰², and R¹⁰² is methyl or ethyl.

12. The compound of claim 11, wherein R^{a} and R^{b}, taken together with the nitrogen to which they are attached, form N-morpholinyl or N-piperidinyl.

13. The compound of claim 7, wherein R^{a} and R^{b} individually are hydrogen or a C1-C3 alkyl optionally substituted with —OH, —SH, halogen, cyano, nitro, amino, —COOH, or C1-C3 alkyl.

14. The compound of claim 13, represented by the following structure:

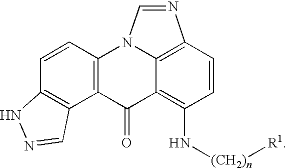

15. The compound of claim 14, wherein n is 2 or 3.

16. The compound of claim 15, wherein R^{a} and R^{b} individually are H, methyl or ethyl.

17. The compound of claim 1 selected from 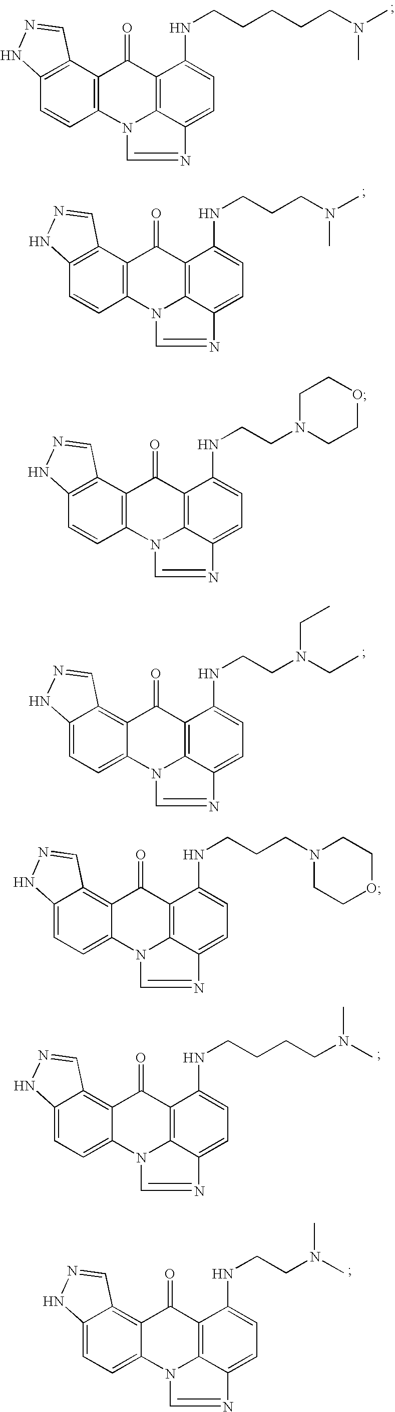 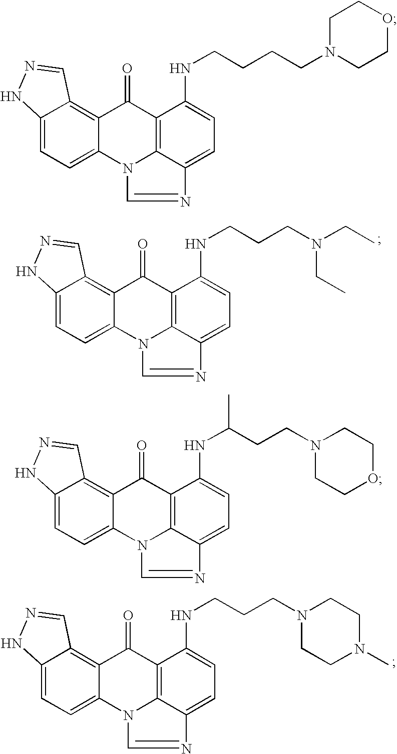
or a pharmaceutically acceptable salts thereof.

18. The compound of claim 1 selected from 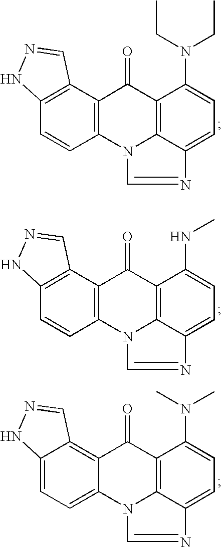
or a pharmaceutically acceptable salts thereof.

19. A method of treating an inflammatory disorder, a demyelinating disorder, a cancer, or a leukemia in a patient, comprising administering to said patient a therapeutically effective amount of a compound of claim 1.

20. The method of claim 19, wherein the disorder is a cancer selected from breast cancer, colorectal cancer, non-small cell lung cancer, ovarian, renal, sarcoma, melanoma, head & neck, hepatocellular, thyroid, multidrug-resistant leukemia, lymphoma, multiple myeloma, esophageal, large bowel, pancreatic, mesothelioma, carcinoma, sarcoma and melanoma.

21. The method of claim 19, wherein the disorder is an inflammatory disorder selected from systemic lupus, inflammatory bowl disease, psoriasis, Crohn's disease, rheumatoid arthritis, sarcoid, Alzheimer's disease, insulin dependent diabetes mellitus, atherosclerosis, asthma, spinal cord injury, stroke, a chronic inflammatory demyelinating neuropathy, multiple sclerosis, a congenital metabolic disorder, a neuropathy with abnormal myelination, drug-induced demyelination, radiation induced demyelination, a hereditary demyelinating condition, a prion-induced demyelination, and encephalitis-induced demyelination.

22. The method of claim 19, wherein the disorder is a demyelinating condition selected from multiple sclerosis, a congenital metabolic disorder, a neuropathy with abnormal myelination, drug-induced demyelination, radiation induced demyelination, a hereditary demyelination condition, a prion-induced demyelination, encephalitis-induced demyelination, a spinal cord injury, Alzheimer's disease, Chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

23. The method of claim 19, wherein the disorder is rheumatoid arthritis or multiple sclerosis.

24. The method of claim 19, wherein the disorder is Crohn's disease, ulcerative colitis, or inflammatory bowel disease.

25. The method of claim 19, wherein the disorder is leukemia.

26. The method of claim 25, wherein the leukemia is selected from acute myeloid leukemia (AML), acute lymphocytic leukemia (ALL), chronic myeloid leukemia (CML) and chronic lymphocytic leukemia (CLL).

27. The method of claim 25, wherein the leukemia is acute myeloid leukemia characterized by a FLT3 mutation.

28. A method of treating a disease or disorder mediated by one or more of a kinase selected from FLT3, CSF-1R, and MYLK2, comprising administering to said patient a therapeutically effective amount of a compound of claim 1.

29. The method of claim 28, wherein the disorder is a FLT3-mediated disorder selected from axonal degeneration, acute transverse myelitis, amyotrophic lateral sclerosis, infantile spinal muscular atrophy, juvenile spinal muscular atrophy, Creutzfeldt-Jakob disease, subacute sclerosing panencephalitis, organ rejection, bone marrow transplant rejection, non-myeloablative bone marrow transplant rejection, ankylosing spondylitis, aplastic anemia, Behcet's disease, graft-versus-host disease, Graves' disease, autoimmune hemolytic anemia, Wegener's granulomatosis, hyper IgE syndrome, idiopathic thrombocytopenia purpura, and Myasthenia gravis.

30. The method of claim 28, wherein the disorder is a CSF-1R-mediated disorder selected from a cardiovascular disease, diseases with an inflammatory component including glomerulonephritis, prosthesis failure, sarcoidosis, congestive obstructive pulmonary disease, asthma, pancreatitis, HIV infection, psoriasis, diabetes, tumor related angiogenesis, age-related macular degeneration, diabetic retinopathy, restenosis, schizophrenia, skeletal pain caused by tumor metastasis or osteoarthritis, or visceral, inflammatory, and neurogenic pain, osteoporosis, Paget's disease, prosthesis failure, osteolytic sarcoma, myeloma, and tumor metastasis to bone, uterine cancer, stomach cancer, hairy cell leukemia, Sjogren's syndrom, uveitis, osteolytic sarcoma, uterine cancer, and stomach cancer.

31. The method of claim 28, wherein the disorder is a MYLK2-mediated barrier dysfunction disorder selected from diseases with an inflammatory component including ulcerative colitis, Crohn's disease, bowel ischemia, colonic ileus, vasogenic ischemia, focal cerebral ischemia, hemorrhagic or septic shock, virus associated myelopathy, septic encephalopathy, glomerulonephritis, prosthesis failure, graft-versus-host disease, sarcoidosis, congestive obstructive pulmonary disease, asthma, pancreatitis, HIV infection, HIV-associated dementia, psoriasis, atopic dermatitis, diabetes, tumor related angiogenesis, restenosis, skeletal pain caused by tumor metastasis or osteoarthritis, or visceral, inflammatory, and neurogenic pain, osteolytic sarcoma, myeloma, and tumor metastasis to bone, uterine cancer, stomach cancer, hairy cell leukemia, Sjogren's syndrom, uveitis, uterine cancer, and stomach cancer.

32. The method of claim 19, wherein one or more additional pharmaceutical agents is co-administered with a compound of formula (I).

33. A compound represented by the following formula: 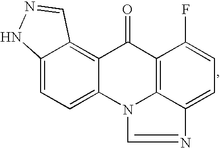
or a salt thereof.

34. A pharmaceutical composition comprising the compound of claim 1 or a pharmaceutically acceptable salt thereof and a pharmaceutically acceptable carrier or excipient.

35. The pharmaceutical composition of claim 32, further comprising an additional therapeutic agent.

36. A method of modulating the activity of FLT3 in a subject, comprising administering to a subject an effective amount of a compound or pharmaceutically acceptable salt as defined in claims 1.

37. A method of modulating the activity of MYLK2 in a subject, comprising administering to a subject an effective amount of a compound or pharmaceutically acceptable salt as defined in any one of claim 1.

38. The compound of claim 1, selected from the group consisting of:

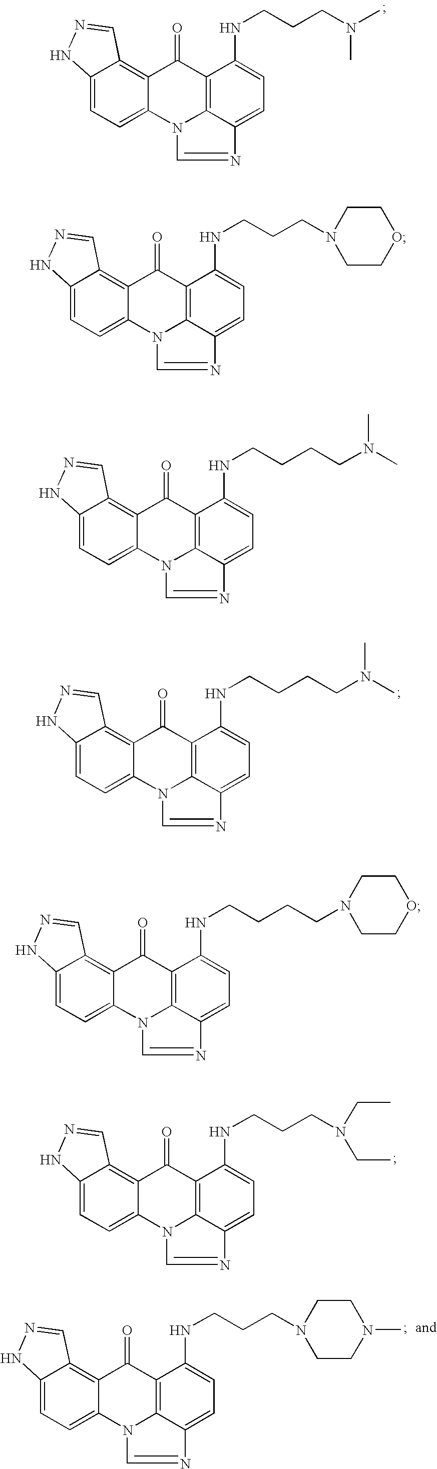

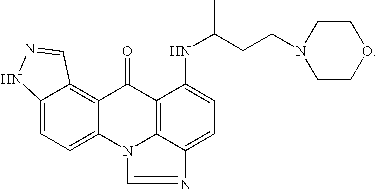

39. A method of treating bone metastases in a patient, comprising administering to said patient a therapeutically effective amount of a compound of claim 1.

40. The method of claim 39, wherein the compound if selected from the group consisting of: [chemical structure]


SEQUENCE LISTING (5 sequences)

SEQ ID NO: 1 - protein, 20 aa, Artificial Sequence
  T-cell receptor (TCR) VBeta6 CDR2 peptide vaccine consisting of TCR VBeta6, amino acid positions 39-58
LGQGPEFLTYFQNEAQLEKS

SEQ ID NO: 2 - protein, 21 aa, Artificial Sequence
  Myelin basic protein immunogen peptide, amino acid positions 79-95
KSHGRTQDENPVVHFFKNIVT

SEQ ID NO: 3 - protein, 17 aa, Artificial Sequence
  Tiplimotide, myelin basic protein immunogen vaccine peptide, amino acid positions 83-99
XKPVVHLFADIVTPRTP

SEQ ID NO: 4 - protein, 17 aa, Artificial Sequence
  Myelin basic protein immunogen peptide, amino acid positions 82-98
DEDPVVHFFKDIVTPRT

SEQ ID NO: 5 - protein, 39 aa, Homo sapiens
SYSMEHFRWGKPVGKKRRPVKVYPDGAEDELAEAFPLEF